SEQUENCE LISTING (85 sequences)

SEQ ID NO: 1 - protein, 224 aa, Microcystis aeruginosa
MVHIYSCQLELHDSLYYATREIGRLYESEPVIHNYALCYALGLVNSDSYRYFCSEQIPQYQEHLNPLNEEKIYVTPARAIAHTAVLNTWKYANNNYHVEMEKTQKNIPSFGRAKEIAPESIFECFIISHHPLQLPKWIRLGKWMSKAEVKLTELSLSKQKEDLFIYPYPLNPLDVMFTHQVIGYDVINMPPVSLIRNVRMRGEYYQISDRPDLKIPARLSYHFG

SEQ ID NO: 2 - protein, 277 aa, Microcystis aeruginosa
MPYSLVLNLTPRSPIYPNFLTGRHLHALFLTLVSSVDQELGNILHTAEADKAFTLSPLQMQSGGKTINSPQWRHEREIASETPCWWRISLLDDRLFGKLTSLWLNLNPKQPWHLGSADLVITSVLATPQSVQPWANSCTYQYLYENASETNREFDFLFATPVTFRQGKFDSALPTRELVFNSLLGRWNRYSGIPFDSIALESIFPSFFDIQTKLADEAYKNQSIGCVGEIHYRLLGEVEPAKIKAINALADFALYAGVGRKTTMGMGMTRRISKDKR

SEQ ID NO: 3 - protein, 332 aa, Microcystis aeruginosa
TFLTSVDAKFFHSEIPYKPMGKYVHFLTIRVTESYPLFQTDGELNKARVRAGIDSKKTISRLSMFKRKQSTPERLVGRELLRNYGLITAEECEYNVKFAMNNADCIIYGFAIGDSGSEKSKVVVDTAFSITPFDESHESFTLNAPYENGTMASKGENNTKVGEVTSRINQQDHIRPQVFFPSIVTLKDPTEASFLYVFNNILRTRHYGAQTTRTGRVRNELIGVIFADGEIVSNLRWTQAIYDRLPDEVLHSIDPLDEDLVMEKATEAIQALMAEEFIVHTDFIGENFQPLLTEVKTLTGTEAGILSVLDQANKESKKYFEQYIEKKKAEKK

SEQ ID NO: 4 - protein, 721 aa, Microcystis aeruginosa
MGNYQVTLKPVYSCPADEIPDGIKVPQGWRLSWHQVETWKALNDPDIDVIFNTAMTGDGKSLAAYLRTLQGYFPIMGLYPTNELARDQRGQIEAYIQRFQPTDQPRVNLLTGPELELYAERDGKTKAIALETRSKQSEILLTNPDIFHYLHRAAYLTPYDNPDQLWNRIDKHFDLFLFDEFHVFGTPQVASIINTMLLIRRANRGKRYLFLSATPDEGLLKRLDKAGFRYRSIDPVREGKYRFPDTPEEANSLAQQGWRQVTSEIELSFIPLPSSFQTSENWLKENKERILDYFKRYPGSKGAIILNSIASVKRLLPIFRELLATIGLTVGENTGLSGTREKLASLNRDLVIGTSTIDVGVDFKINFLIFESSDAGNFIQRFGRLGRHSGYDRKGTAVKFTNFTAIALVPKFFLERLFEKKDAPLQVGERYDRIQLQEAIKSNYRHINNFEGYYQRWGAVQSFQLWWNLGSPKIKSQYGESRQKFQQECEEVFDTSLKRVAGRVKGWADEWKELSGKNGNPIFEDASSFRGSSPLLCGLYDSTEPEECDRFKTYDLPSILGNLEVEVWRKGEFKRQIEATKTPIARRRFDYCLAFLNLKGYREERLNWRFTYDGDLGEIASAWKVQVLTGIGVWQPDNPWLDRISRELRDLALVSFVFAYPVAAVRQRLQLPMHFGIYPISDESSLHSPLSPYSIAIGQAALLLDTLAHRFKGKGGEVWIC

SEQ ID NO: 5 - protein, 1156 aa, Microcystis aeruginosa
MPKKQKKLEETGQLNLFDNTTEIDDEDLDFEFEDIDLESLVSEDLGITESVSDRRVETVRQLLTLKLLREAIRAENPDDRVMADFAEMVLPNLLRLAIGVTAKGGNFIEAVDRGRELRNKPKAKRDNAGDQSLNTHLLNGLFPANLIEKRLQKLNTTVRRIIKEFERRLAIAGFLVHDFEKFSYDRFPSMSERYIQIQRDFIQDPFKNQDPRKLSREEHREILQVLIPELGLDRFLFPDNPERWLEYLDDLLYIAKNTQRRNDTDLNTSEDGLNVRLNDRVIESLCDLACLADRLASIIKHPHDAEKASLQDLLYSLSDGELKFTYHSIAENRGVLTNVLNNAVMEAHQELDYQPLLYLPTGVVYIAPKNAPEVSLETLPNRVVDTIKSLCSGELQRKQTGFGRDGKGMKYADYYSQFFDDAGLMRAALNATLRILGDNKASVARSRGENLIKFQQQGVLPTDYDFHCEDDIRIDRLAEFGDVVTRKIWGDRLEKIEQARKLQKNLPAPPDLDLISEIAHYWNLENYLPQIRAIKRINESLKELKLKGNTGGVPYEWYYLAAQYLKQHPGIEDIRPVAEDLIAFLAAKIAAIVAGYNLPDGWEDLREWVNQVVQLPGRELAHSIETFQKELNHYNAAKKQGRGRQLLCSISHSPYSVSEQMESAVLFTPQVYTNKQMLAGSNAKRNISSIAGTEMMLRQILMNQTQAVGKRFEDGKYRYLYFYPTYYFTPETNSFLQKAYANIAQTRFDSSIKLHFVDKNLVANFDRTRYQSVDSFLIDEKLRQKKETINEEEDGKKDRTFKLSYPEDKPLTFYFMALPPGRNPTDTESWVMPAWLGLAFPMILDVKTVVSESPIPPYRDGAEFEETVFLDSAPQAIRSLTRCDRFRLDRVLNPWQDNDGKKYSAPLNTLTAAYSIHLDVNSKQGKTGYDPNWGKLTELAINLETSPLYVFHYFKQWKRGKDADIPSANRIALYLYDFYPCFDPYVQANRTNLTIDMTAESPLNHPKNLTELYRQFYRAKSSKGKPIKANAILKPIDEAADIILKADKAISDDLTSLVAARLFKLMDRVRSQTAEGRYVIKERDQEREKILDFAKYFVKNVFEESFESDRARLAGRQLNIIRDTCEFLYRLEMDKERRQRQVQPLDTSNSSSEEEE

SEQ ID NO: 6 - RNA, 109 nt, synthetic construct
  misc_feature 1..109: note = TiDcrRNA
  misc_difference 38..72: note = n is a, c, g, or u
  source 1..109: mol_type = other RNA
gttccaattaatcttaagccctattagggattgaaacnnnnnnnnnnnnnnnnnnnnnnnnnnnnnnnnnnngttccaattaatcttaagccctattagggattgaaac

SEQ ID NO: 7 - DNA, 675 bp, synthetic construct
  misc_feature 1..675: note = Cas5d nucleotide sequence for expression in Escherichia coli
  source 1..675: mol_type = other DNA
atggtgcatatttatagctgccagctggaactgcatgatagcctgtattatgcgacccgcgaaattggccgcctgtatgaaagcgaaccggtgattcataactatgcgctgtgctatgcgctgggcctggtgaacagcgatagctatcgctatttctgcagcgaacagattccgcagtatcaggaacatctgaacccgctgaacgaagaaaaaatttatgtgaccccggcgcgcgcgattgcgcataccgcggtgctgaacacctggaaatatgcgaacaacaactatcatgtggaaatggaaaaaacccagaaaaacattccgagcttcggccgcgcgaaagaaattgcgccggaaagcattttcgaatgcttcattattagccatcatccgctgcagctgccgaaatggattcgcctgggcaaatggatgagcaaagcggaagtgaaactgaccgaactgagcctgagcaaacagaaagaagatctgttcatttatccgtatccgctgaacccgctggatgtgatgttcacccatcaggtgattggctatgatgtgattaacatgccgccggtgagcctgattcgcaacgtgcgcatgcgcggcgaatattatcagattagcgatcgcccggatctgaaaattccggcacgtctgagctatcatttcggctaa

SEQ ID NO: 8 - DNA, 834 bp, synthetic construct
  misc_feature 1..834: note = Cas6d nucleotide sequence for expression in Escherichia coli
  source 1..834: mol_type = other DNA
atgccgtatagcctggtgctgaacctgaccccgcgcagcccgatttatccgaacttcctgaccggccgccatctgcatgcgctgttcctgaccctggtgagcagcgtggatcaggaactgggcaacattctgcataccgcggaagcggataaagcgttcaccctgagcccgctgcagatgcagagcggcggcaaaaccattaacagcccgcagtggcgccatgaacgcgaaattgcgagcgaaaccccgtgctggtggcgcattagcctgctggatgatcgcctgttcggcaaactgaccagcctgtggctgaacctgaacccgaaacagccgtggcatctgggcagcgcggatctggtgattaccagcgtgctggcgaccccgcagagcgtgcagccgtgggcgaacagctgcacctatcagtatctgtatgaaaacgcgagcgaaaccaaccgcgaattcgatttcctgttcgcgaccccggtgaccttccgccagggcaaattcgatagcgcgctgccgacccgcgaactggtgttcaacagcctgctgggccgctggaaccgctatagcggcattccgttcgatagcattgcgctggaaagcattttcccgagcttcttcgatattcagaccaaactggcggatgaagcgtataaaaaccagagcattggctgcgtgggcgaaattcattatcgcctgctgggcgaagtggaaccggcgaaaattaaagcgattaacgcgctggcggatttcgcgctgtatgcgggcgtgggccgcaaaaccaccatgggcatgggcatgacccgccgcattagcaaagataaacgctaa

SEQ ID NO: 9 - DNA, 1002 bp, synthetic construct
  misc_feature 1..1002: note = Cas7d nucleotide sequence for expression in Escherichia coli
  source 1..1002: mol_type = other DNA
atgaccttcctgaccagcgtggatgcgaaattcttccatagcgaaattccgtataaaccgatgggcaaatatgtgcatttcctgaccattcgcgtgaccgaaagctatccgctgttccagaccgatggcgaactgaacaaagcgcgcgtgcgcgcgggcattgacagcaagaaaaccattagccgcctgagcatgttcaaacgcaaacagagcaccccggaacgcctggtgggccgcgaactgctgcgcaactatggcctgattaccgcggaagaatgcgaatataacgtgaaattcgcgatgaacaacgcggattgcattatttatggcttcgcgattggcgatagcggcagcgaaaaaagcaaagtggtggtggataccgcgttcagcattaccccgttcgatgaaagccatgagagcttcaccctgaacgcgccgtatgaaaacggcacgatggcgagcaaaggcgaaaacaacaccaaagtgggcgaagtgaccagccgcattaaccagcaggatcatattcgcccgcaggtgttcttcccgagcattgtgaccctgaaagatccgaccgaagcgagcttcctgtatgtgttcaacaacattctgcgcacccgccattatggcgcgcagaccacccgcaccggccgcgtgcgcaacgaactgattggcgtgattttcgcggatggcgaaattgtgagcaacctgcgctggacccaggcgatttatgatcgcctgccggatgaagtgctgcatagcattgatccgctggatgaagatctggtgatggaaaaagcgaccgaagcgattcaggcgctgatggcggaagaatttattgtgcataccgatttcattggcgaaaacttccagccgctgctgaccgaagtgaaaaccctgaccggcaccgaagcgggcattctgagcgtgctggatcaggcgaacaaagaaagcaaaaaatatttcgaacagtatattgaaaagaaaaaggcggaaaagaaataa

SEQ ID NO: 10 - DNA, 2166 bp, synthetic construct
  misc_feature 1..2166: note = Cas3d nucleotide sequence for expression in Escherichia coli
  source 1..2166: mol_type = other DNA
atgggaaactatcaggtgactcttaagccagtgtattcttgcccagctgacgagatcccagacggaatcaaggtgccacagggatggagactttcttggcaccaggtggagacttggaaggctcttaacgacccagacatcgacgtgatcttcaacactgctatgactggagacggaaagtctcttgctgcttatcttagaactcttcagggatatttcccaatcatgggactttatccaactaacgagcttgctagagatcagagaggacagatcgaggcttatatccagagattccagccaactgaccagccaagagtgaaccttcttactggaccagagcttgagctttatgctgagagagacggaaagactaaggctatcgctcttgagactagatctaagcagtctgagatccttcttactaacccagacatcttccactatcttcacagagctgcttatcttactccatatgacaacccagaccagctttggaacagaatcgacaagcacttcgaccttttccttttcgacgagttccacgtgttcggaactccacaggtggcttctatcatcaacactatgcttcttatcagaagagctaacagaggaaagagatatcttttcctttctgctactccagacgagggacttcttaagagacttgacaaggctggattcagatatagatctatcgacccagtgagagagggaaagtatagattcccagacactccagaggaggctaactctcttgctcagcagggatggagacaggtgacttctgagatcgagctttctttcatcccacttccatcttctttccagacttctgagaactggcttaaggagaacaaggagagaatccttgactatttcaagagatatccaggatctaagggagctatcatccttaactctatcgcttctgtgaagagacttcttccaatcttcagagagcttcttgctactatcggacttactgtgggagagaacactggactttctggaactagagagaaacttgcttctcttaacagagatcttgtgatcggaacttctactatcgacgtgggagtggacttcaagatcaacttccttatcttcgagtcttctgacgctggaaacttcatccagagattcggaagacttggaagacactctggatatgacagaaagggaactgctgtgaagttcactaacttcactgctatcgctcttgtgccaaagttcttccttgagagacttttcgagaagaaggacgctccacttcaggtgggagagagatatgacagaatccagcttcaggaggctatcaagtctaactatagacacatcaacaacttcgagggatattatcagagatggggagctgtgcagtctttccagctttggtggaaccttggatctccaaagatcaagtctcagtatggagagtcaagacagaagttccagcaggagtgcgaggaggtgttcgacacttctcttaagagagtggctggaagagtgaagggatgggctgacgagtggaaggagctttctggaaagaacggaaacccaatcttcgaggacgcttcttctttcagaggatcttctccacttctttgcggactttatgactctactgagccagaggagtgcgacagattcaagacttatgaccttccatctatccttggaaaccttgaggtggaggtgtggagaaagggagagttcaagagacagatcgaggctactaagactccaatcgctagaagaagattcgactattgccttgctttccttaaccttaagggatatagagaggagagacttaactggagattcacttatgacggagatcttggagagatcgcttctgcttggaaggtgcaggtgcttactggaatcggagtgtggcagccagacaacccttggcttgacagaatctcaagagagcttagagatcttgctcttgtgtctttcgtgttcgcttatccagtggctgctgtgagacagagacttcagcttccaatgcacttcggaatctatccaatctctgacgagtcttctcttcactctccactttctccatattctatcgctatcggacaggctgctcttcttcttgacactcttgctcacagattcaagggaaagggaggagaggtgtggatctgctag

SEQ ID NO: 11 - DNA, 3471 bp, synthetic construct
  misc_feature 1..3471: note = Cas10d nucleotide sequence for expression in Escherichia coli
  source 1..3471: mol_type = other DNA
atgccaaagaagcagaagaagcttgaggagactggacagcttaaccttttcgacaacactactgagatcgacgacgaggaccttgacttcgagttcgaggacatcgaccttgagtctcttgtgtctgaggaccttggaatcactgagtctgtgtctgacagaagagtggagactgtgagacagcttcttactcttaagcttcttagagaggctatcagagctgagaacccagacgacagagtgatggctgacttcgctgagatggtgcttccaaaccttcttagacttgctatcggagtgactgctaagggaggaaacttcatcgaggctgtggacagaggaagagagcttagaaacaagccaaaggctaagagagacaacgctggagatcagtctcttaacactcaccttcttaacggacttttcccagctaaccttatcgagaagagacttcagaagcttaacactactgtgagaagaatcatcaaggagttcgagagaagacttgctatcgctggattccttgtgcacgacttcgagaagttctcttatgacagattcccatctatgtctgagagatatatccagatccagagagacttcatccaggacccattcaagaaccaggacccaagaaagctttcaagagaggagcacagagagatccttcaggtgcttatcccagagcttggacttgacagattccttttcccagacaacccagagagatggcttgagtatcttgacgaccttctttatatcgctaagaacactcagagaagaaacgacactgaccttaacacttctgaggacggacttaacgtgagacttaacgacagagtgatcgagtctctttgcgaccttgcttgccttgctgacagacttgcttctatcatcaagcacccacacgacgctgagaaggcttctcttcaggaccttctttattctctttctgacggagagcttaagttcacttatcactctatcgctgagaacagaggagtgcttactaacgtgcttaacaacgctgtgatggaggctcaccaggagcttgactatcagccacttctttatcttccaactggagtggtgtatatcgctccaaagaacgctccagaggtgtctcttgagactcttccaaacagagtggtggacactatcaagtctctttgctctggagagcttcagagaaagcagactggattcggaagagacggaaagggtatgaagtatgctgactattattctcagttcttcgacgacgctggacttatgagagctgctcttaacgctactcttagaatccttggagacaacaaggcttctgtggctagatctagaggagagaaccttatcaagttccagcagcagggagtgcttccaactgactatgacttccactgcgaggacgacatcagaatcgacagacttgctgagttcggagacgtggtgactagaaagatctggggagacagacttgagaagatcgagcaggctagaaagcttcagaagaaccttccagctccaccagaccttgaccttatctctgagatcgctcactattggaaccttgagaactatcttccacagatcagagctatcaagagaatcaacgagtctcttaaggagcttaagcttaagggaaacactggaggagtgccatatgagtggtattatcttgctgctcagtatcttaagcagcacccaggaatcgaggacatcagaccagtggctgaggaccttatcgctttccttgctgctaagatcgctgctatcgtggctggatataaccttccagacggatgggaggaccttagagagtgggtgaaccaggtggtgcagcttccaggaagagagcttgctcactctatcgagactttccagaaggagcttaaccactataacgctgctaagaagcagggaagaggaagacagcttctttgctctatctctcactctccatattctgtgtctgagcagatggagtctgctgtgcttttcactccacaggtgtatactaacaagcagatgcttgctggatctaacgctaagagaaacatctcttctatcgctggaactgagatgatgcttagacagatccttatgaaccagactcaggctgtgggaaagagattcgaggacggaaagtatagatatctttatttctatccaacttattatttcactccagagactaactctttccttcagaaggcttatgctaacatcgctcagactagattcgactcttctatcaagcttcacttcgtggacaagaaccttgtggctaacttcgacagaactagatatcagtctgtggactctttccttatcgacgagaagcttagacagaagaaggagactatcaacgaggaggaggacggaaagaaggacagaactttcaagctttcttatccagaggacaagccacttactttctatttcatggctcttccaccaggaagaaacccaactgacactgagtcttgggtaatgccagcttggcttggacttgctttcccaatgatccttgacgtgaagactgtggtgtctgagtctccaatcccaccatatagagacggagctgagttcgaggagactgtgttccttgactctgctccacaggctatcagatctcttactagatgcgacagattcagacttgacagagtgcttaacccttggcaggacaacgacggaaagaagtattctgctccacttaacactcttactgctgcttattctatccaccttgacgtgaactctaagcagggaaagactggatatgacccaaactggggaaagcttactgagcttgctatcaaccttgagacttctccactttatgtgttccactatttcaagcagtggaagagaggaaaggacgctgacatcccatctgctaacagaatcgctctttatctttatgacttctatccatgcttcgacccatatgtgcaggctaacagaactaaccttactatcgacatgactgctgagtctccacttaaccacccaaagaaccttactgagctttatagacagttctatagagctaagtcttctaagggaaagccaatcaaggctaacgctatccttaagccaatcgacgaggctgctgacatcatccttaaggctgacaaggctatctctgacgaccttacttctcttgtggctgctagacttttcaagcttatggacagagtgagatctcagactgctgagggaagatatgtgatcaaggagagagatcaggagagagagaagatccttgacttcgctaagtatttcgtgaagaacgtgttcgaggagtctttcgagtctgacagagctagacttgctggaagacagcttaacatcatcagagacacttgcgagttcctttatagacttgagatggacaaggagagaagacagagacaggtgcagccacttgacacttctaactcttcttctgaggaggaggagtaa

SEQ ID NO: 12 - DNA, 35 bp, synthetic construct
  misc_feature 1..35: note = J23108 synthesis promoter
  source 1..35: mol_type = other DNA
ctgacagctagctcagtcctaggtataatgctagc

SEQ ID NO: 13 - DNA, 36 bp, synthetic construct
  misc_feature 1..36: note = Ribosomal binding sequence
  source 1..36: mol_type = other DNA
aataattttgtttaactttaagaaggagatatacat

SEQ ID NO: 14 - DNA, 24 bp, synthetic construct
  misc_feature 1..24: note = Terminator sequence STOP767
  source 1..24: mol_type = other DNA
agatcctgtaaaacgacggccagt

SEQ ID NO: 15 - DNA, 19 bp, synthetic construct
  misc_feature 1..19: note = Terminator sequence STOP768(1)
  source 1..19: mol_type = other DNA
cgccagggttttcccagtc

SEQ ID NO: 16 - DNA, 19 bp, synthetic construct
  misc_feature 1..19: note = Terminator sequence TOP768(2)
  source 1..19: mol_type = other DNA
cgccagggttttcccagtc

SEQ ID NO: 17 - DNA, 47 bp, synthetic construct
  misc_feature 1..47: note = T7 terminator sequence
  source 1..47: mol_type = other DNA
tagcataaccccttggggcctctaaacgggtcttgaggggttttttg

SEQ ID NO: 18 - DNA, 37 bp, synthetic construct
  misc_feature 1..37: note = CRISPR repeat sequence
  source 1..37: mol_type = other DNA
gttccaattaatcttaagccctattagggattgaaac

SEQ ID NO: 19 - DNA, 19 bp, synthetic construct
  misc_feature 1..19: note = T7 promoter sequence
  source 1..19: mol_type = other DNA
taatacgactcactatagg

SEQ ID NO: 20 - DNA, 109 bp, synthetic construct
  misc_feature 1..109: note = crRNA expression cassette
  source 1..109: mol_type = other DNA
gttccaattaatcttaagccctattagggattgaaacggtaataatacgactcactatagggagaaaggatcgttccaattaatcttaagccctattagggattgaaac

SEQ ID NO: 21 - DNA, 581 bp, synthetic construct
  misc_feature 1..581: note = Synthesis cccdB gene expression cassette
  source 1..581: mol_type = other DNA
aggctttaatacgactcactatagggagaaaggatccataaaggaggtaaataatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataa

SEQ ID NO: 22 - protein, 18 aa, synthetic construct
  REGION 1..18: note = Nuclear localizing signal (NLS) amino acid sequence
DPKKKRKVDPKKKRKVSG

SEQ ID NO: 23 - DNA, 54 bp, synthetic construct
  misc_feature 1..54: note = NLS nucleotide sequence
  source 1..54: mol_type = other DNA
gacccaaagaagaagcggaaggtagaccctaagaagaagcgcaaggtttctgga

SEQ ID NO: 24 - protein, 20 aa, synthetic construct
  REGION 1..20: note = Self-cleaving peptide 2A amino acid sequence
GSEGRGSLLTCGDVEENPGP

SEQ ID NO: 25 - DNA, 60 bp, synthetic construct
  misc_feature 1..60: note = Self-cleaving peptide 2A(1) coding sequence
  source 1..60: mol_type = other DNA
ggctctgagggcagaggcagcctgctgacctgcggcgacgtggaggaaaaccctggccct

SEQ ID NO: 26 - DNA, 60 bp, synthetic construct
  misc_feature 1..60: note = Self-cleaving peptide 2A(2) coding sequence
  source 1..60: mol_type = other DNA
gggtctgagggacgcggctccctgctcacctgtggagatgtggaagagaacccaggcccc

SEQ ID NO: 27 - DNA, 60 bp, synthetic construct
  misc_feature 1..60: note = Self-cleaving peptide 2A(3) coding sequence
  source 1..60: mol_type = other DNA
ggttctgaaggcagaggctctctgctgacatgtggggatgtggaggaaaatcctggccct

SEQ ID NO: 28 - DNA, 60 bp, synthetic construct
  misc_feature 1..60: note = Self-cleaving peptide 2A(4) coding sequence
  source 1..60: mol_type = other DNA
ggatccgagggcagaggaagtctgctaacatgcggtgacgttgaggagaatcccgggcca

SEQ ID NO: 29 - DNA, 820 bp, synthetic construct
  misc_feature 1..820: note = 2 x cauliflower mosaic virus 35S gene promoter + omega sequence
  source 1..820: mol_type = other DNA
gccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggccggtctagagtatttttacaacaattaccaacaacaacaaacaacaaacaacattacaattactatttacaatt

SEQ ID NO: 30 - DNA, 447 bp, synthetic construct
  misc_feature 1..447: note = Arabidopsis shock protein 18.2kDa gene terminator
  source 1..447: mol_type = other DNA
atatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagatgggctcatagagtaaaacgaggcgagggacctataaacctcccttcatcatgctatttcatgatctattttataaaataaagatgtagaaaaaagtaagcgtaataaccgcaaaacaaatgatttaaaacatggcacataatgaggagattaagttcggtttacgtttattttagtactaattgtaacgtgagac

SEQ ID NO: 31 - DNA, 97 bp, synthetic construct
  misc_feature 1..97: note = crRNA expression cassette
  source 1..97: mol_type = other DNA
gttccaattaatcttaagccctattagggattgaaacggagaccctcaattgtcggtctcgttccaattaatcttaagccctattagggattgaaac

SEQ ID NO: 32 - DNA, 448 bp, synthetic construct
  misc_feature 1..448: note = Arabidopsis U6 snRNA-26 gene promoter sequence
  source 1..448: mol_type = other DNA
aagcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgatt

SEQ ID NO: 33 - DNA, 723 bp, synthetic construct
  misc_feature 1..723: note = 2xNLS + Cas5d
  source 1..723: mol_type = other DNA
gacccaaagaagaagcggaaggtagaccctaagaagaagcgcaaggtttctggagtgcacatctattcttgccagcttgagcttcacgactctctttattatgctactagagagatcggaagactttatgagtctgagccagtgatccacaactatgctctttgctatgctcttggacttgtgaactctgactcttatagatatttctgctctgagcagatcccacagtatcaggagcaccttaacccacttaacgaggagaagatctatgtgactccagctagagctatcgctcacactgctgtgcttaacacttggaagtatgctaacaacaactatcacgtggagatggagaagactcagaagaacatcccatctttcggaagagctaaggagatcgctccagagtctatcttcgagtgcttcatcatctctcaccacccacttcagcttccaaagtggatcagacttggaaagtggatgtctaaggctgaggtgaagcttactgagctttctctttctaagcagaaggaggaccttttcatctatccatatccacttaacccacttgacgtgatgttcactcaccaggttatcggatatgacgtgatcaacatgccaccagtgtctcttatcagaaacgtgagaatgagaggagagtattatcagatctctgacagaccagaccttaagatcccagctagactttcttatcacttcgga

SEQ ID NO: 34 - DNA, 882 bp, synthetic construct
  misc_feature 1..882: note = 2xNLS + Cas6d
  source 1..882: mol_type = other DNA
gacccaaagaagaagcggaaggtagaccctaagaagaagcgcaaggtttctggaccatattctcttgtgcttaaccttactccaagatctccaatctatccaaacttccttactggaagacaccttcacgctcttttccttactcttgtgtcttctgtggaccaggagcttggaaacatccttcacactgctgaggctgacaaggctttcactctttctccacttcagatgcagtctggaggaaagactatcaactctccacagtggagacacgagagagagatcgcttctgagactccatgctggtggagaatctctcttcttgacgacagacttttcggaaagcttacttctctttggcttaaccttaacccaaagcagccttggcaccttggatctgctgaccttgtgatcacttctgtgcttgctactccacagtctgtgcagccttgggctaactcttgcacttatcagtatctttatgagaacgcttctgagactaacagagagttcgacttccttttcgctactccagtgactttcagacagggaaagttcgactctgctcttccaactagagagcttgtgttcaactctcttcttggaagatggaacagatattctggaatcccattcgactctatcgctcttgagtctatcttcccatctttcttcgacatccagactaagcttgctgacgaggcttataagaaccagtctatcggatgcgtgggagagatccactatagacttcttggagaggtggagccagctaagatcaaggctatcaacgctcttgctgacttcgctctttatgctggagtgggaagaaagactactatgggaatgggaatgactagaagaatctctaaggacaagaga

SEQ ID NO: 35 - DNA, 1053 bp, synthetic construct
  misc_feature 1..1053: note = 2xNLS + Cas7d
  source 1..1053: mol_type = other DNA
gacccaaagaagaagcggaaggtagaccctaagaagaagcgcaaggtttctggaactttccttacttctgtggacgctaagttcttccactctgagatcccatataagccaatgggaaagtatgtgcacttccttactatcagagtgactgagtcttatccacttttccagactgacggagagcttaacaaggctagagtgagagctggaatcgactctaagaagactatctcaagactttctatgttcaagagaaagcagtctactccagagagacttgtgggaagagagcttcttagaaactatggacttatcactgctgaggagtgcgagtataacgtgaagttcgctatgaacaacgctgactgcatcatctatggattcgctatcggagactctggatctgagaagtctaaggtggtggtggacactgctttctctatcactccattcgacgagtctcacgagtctttcactcttaacgctccatatgagaacggaactatggcttctaagggagagaacaacactaaggtgggagaggtgacttcaagaatcaaccagcaggaccacatcagaccacaggtgttcttcccatctatcgtgactcttaaggacccaactgaggcttctttcctttatgtgttcaacaacatccttagaactagacactatggtgctcagactactagaactggaagagtgagaaacgagcttatcggagtgatcttcgctgacggagagatcgtgtctaaccttagatggactcaggctatctatgacagacttccagacgaggtgcttcactctatcgacccacttgacgaggaccttgtgatggagaaggctactgaggctatccaggctcttatggctgaggagttcatcgtgcacactgacttcatcggagagaacttccagccacttcttactgaggtgaagactcttactggaactgaggctggaatcctttctgtgcttgaccaggctaacaaggagtctaagaagtatttcgagcagtatatcgagaagaagaaggctgagaagaagtaa

SEQ ID NO: 36 - DNA, 2217 bp, synthetic construct
  misc_feature 1..2217: note = 2xNLS + Cas3d
  source 1..2217: mol_type = other DNA
atggacccaaagaagaagcggaaggtagaccctaagaagaagcgcaaggtttctggaggaaactatcaggtgactcttaagccagtgtattcttgcccagctgacgagatcccagacggaatcaaggtgccacagggatggagactttcttggcaccaggtggagacttggaaggctcttaacgacccagacatcgacgtgatcttcaacactgctatgactggagacggaaagtctcttgctgcttatcttagaactcttcagggatatttcccaatcatgggactttatccaactaacgagcttgctagagatcagagaggacagatcgaggcttatatccagagattccagccaactgaccagccaagagtgaaccttcttactggaccagagcttgagctttatgctgagagagacggaaagactaaggctatcgctcttgagactagatctaagcagtctgagatccttcttactaacccagacatcttccactatcttcacagagctgcttatcttactccatatgacaacccagaccagctttggaacagaatcgacaagcacttcgaccttttccttttcgacgagttccacgtgttcggaactccacaggtggcttctatcatcaacactatgcttcttatcagaagagctaacagaggaaagagatatcttttcctttctgctactccagacgagggacttcttaagagacttgacaaggctggattcagatatagatctatcgacccagtgagagagggaaagtatagattcccagacactccagaggaggctaactctcttgctcagcagggatggagacaggtgacttctgagatcgagctttctttcatcccacttccatcttctttccagacttctgagaactggcttaaggagaacaaggagagaatccttgactatttcaagagatatccaggatctaagggagctatcatccttaactctatcgcttctgtgaagagacttcttccaatcttcagagagcttcttgctactatcggacttactgtgggagagaacactggactttctggaactagagagaaacttgcttctcttaacagagatcttgtgatcggaacttctactatcgacgtgggagtggacttcaagatcaacttccttatcttcgagtcttctgacgctggaaacttcatccagagattcggaagacttggaagacactctggatatgacagaaagggaactgctgtgaagttcactaacttcactgctatcgctcttgtgccaaagttcttccttgagagacttttcgagaagaaggacgctccacttcaggtgggagagagatatgacagaatccagcttcaggaggctatcaagtctaactatagacacatcaacaacttcgagggatattatcagagatggggagctgtgcagtctttccagctttggtggaaccttggatctccaaagatcaagtctcagtatggagagtcaagacagaagttccagcaggagtgcgaggaggtgttcgacacttctcttaagagagtggctggaagagtgaagggatgggctgacgagtggaaggagctttctggaaagaacggaaacccaatcttcgaggacgcttcttctttcagaggatcttctccacttctttgcggactttatgactctactgagccagaggagtgcgacagattcaagacttatgaccttccatctatccttggaaaccttgaggtggaggtgtggagaaagggagagttcaagagacagatcgaggctactaagactccaatcgctagaagaagattcgactattgccttgctttccttaaccttaagggatatagagaggagagacttaactggagattcacttatgacggagatcttggagagatcgcttctgcttggaaggtgcaggtgcttactggaatcggagtgtggcagccagacaacccttggcttgacagaatctcaagagagcttagagatcttgctcttgtgtctttcgtgttcgcttatccagtggctgctgtgagacagagacttcagcttccaatgcacttcggaatctatccaatctctgacgagtcttctcttcactctccactttctccatattctatcgctatcggacaggctgctcttcttcttgacactcttgctcacagattcaagggaaagggaggagaggtgtggatctgc

SEQ ID NO: 37 - DNA, 3519 bp, synthetic construct
  misc_feature 1..3519: note = 2xNLS + Cas10d
  source 1..3519: mol_type = other DNA
gacccaaagaagaagcggaaggtagaccctaagaagaagcgcaaggtttctggaccaaagaagcagaagaagcttgaggagactggacagcttaaccttttcgacaacactactgagatcgacgacgaggaccttgacttcgagttcgaggacatcgaccttgagtctcttgtgtctgaggaccttggaatcactgagtctgtgtctgacagaagagtggagactgtgagacagcttcttactcttaagcttcttagagaggctatcagagctgagaacccagacgacagagtgatggctgacttcgctgagatggtgcttccaaaccttcttagacttgctatcggagtgactgctaagggaggaaacttcatcgaggctgtggacagaggaagagagcttagaaacaagccaaaggctaagagagacaacgctggagatcagtctcttaacactcaccttcttaacggacttttcccagctaaccttatcgagaagagacttcagaagcttaacactactgtgagaagaatcatcaaggagttcgagagaagacttgctatcgctggattccttgtgcacgacttcgagaagttctcttatgacagattcccatctatgtctgagagatatatccagatccagagagacttcatccaggacccattcaagaaccaggacccaagaaagctttcaagagaggagcacagagagatccttcaggtgcttatcccagagcttggacttgacagattccttttcccagacaacccagagagatggcttgagtatcttgacgaccttctttatatcgctaagaacactcagagaagaaacgacactgaccttaacacttctgaggacggacttaacgtgagacttaacgacagagtgatcgagtctctttgcgaccttgcttgccttgctgacagacttgcttctatcatcaagcacccacacgacgctgagaaggcttctcttcaggaccttctttattctctttctgacggagagcttaagttcacttatcactctatcgctgagaacagaggagtgcttactaacgtgcttaacaacgctgtgatggaggctcaccaggagcttgactatcagccacttctttatcttccaactggagtggtgtatatcgctccaaagaacgctccagaggtgtctcttgagactcttccaaacagagtggtggacactatcaagtctctttgctctggagagcttcagagaaagcagactggattcggaagagacggaaagggtatgaagtatgctgactattattctcagttcttcgacgacgctggacttatgagagctgctcttaacgctactcttagaatccttggagacaacaaggcttctgtggctagatctagaggagagaaccttatcaagttccagcagcagggagtgcttccaactgactatgacttccactgcgaggacgacatcagaatcgacagacttgctgagttcggagacgtggtgactagaaagatctggggagacagacttgagaagatcgagcaggctagaaagcttcagaagaaccttccagctccaccagaccttgaccttatctctgagatcgctcactattggaaccttgagaactatcttccacagatcagagctatcaagagaatcaacgagtctcttaaggagcttaagcttaagggaaacactggaggagtgccatatgagtggtattatcttgctgctcagtatcttaagcagcacccaggaatcgaggacatcagaccagtggctgaggaccttatcgctttccttgctgctaagatcgctgctatcgtggctggatataaccttccagacggatgggaggaccttagagagtgggtgaaccaggtggtgcagcttccaggaagagagcttgctcactctatcgagactttccagaaggagcttaaccactataacgctgctaagaagcagggaagaggaagacagcttctttgctctatctctcactctccatattctgtgtctgagcagatggagtctgctgtgcttttcactccacaggtgtatactaacaagcagatgcttgctggatctaacgctaagagaaacatctcttctatcgctggaactgagatgatgcttagacagatccttatgaaccagactcaggctgtgggaaagagattcgaggacggaaagtatagatatctttatttctatccaacttattatttcactccagagactaactctttccttcagaaggcttatgctaacatcgctcagactagattcgactcttctatcaagcttcacttcgtggacaagaaccttgtggctaacttcgacagaactagatatcagtctgtggactctttccttatcgacgagaagcttagacagaagaaggagactatcaacgaggaggaggacggaaagaaggacagaactttcaagctttcttatccagaggacaagccacttactttctatttcatggctcttccaccaggaagaaacccaactgacactgagtcttgggtaatgccagcttggcttggacttgctttcccaatgatccttgacgtgaagactgtggtgtctgagtctccaatcccaccatatagagacggagctgagttcgaggagactgtgttccttgactctgctccacaggctatcagatctcttactagatgcgacagattcagacttgacagagtgcttaacccttggcaggacaacgacggaaagaagtattctgctccacttaacactcttactgctgcttattctatccaccttgacgtgaactctaagcagggaaagactggatatgacccaaactggggaaagcttactgagcttgctatcaaccttgagacttctccactttatgtgttccactatttcaagcagtggaagagaggaaaggacgctgacatcccatctgctaacagaatcgctctttatctttatgacttctatccatgcttcgacccatatgtgcaggctaacagaactaaccttactatcgacatgactgctgagtctccacttaaccacccaaagaaccttactgagctttatagacagttctatagagctaagtcttctaagggaaagccaatcaaggctaacgctatccttaagccaatcgacgaggctgctgacatcatccttaaggctgacaaggctatctctgacgaccttacttctcttgtggctgctagacttttcaagcttatggacagagtgagatctcagactgctgagggaagatatgtgatcaaggagagagatcaggagagagagaagatccttgacttcgctaagtatttcgtgaagaacgtgttcgaggagtctttcgagtctgacagagctagacttgctggaagacagcttaacatcatcagagacacttgcgagttcctttatagacttgagatggacaaggagagaagacagagacaggtgcagccacttgacacttctaactcttcttctgaggaggaggag

SEQ ID NO: 38 - DNA, 35 bp, Nicotiana benthamiana
  source 1..35: mol_type = unassigned DNA
tgggtggtttgtctacagcaaaatatctggcagat

SEQ ID NO: 39 - DNA, 35 bp, Nicotiana benthamiana
  source 1..35: mol_type = unassigned DNA
aaatttgctattggactcttgccagcaatgcttgg

SEQ ID NO: 40 - DNA, 35 bp, Solanum lycopersicum
  source 1..35: mol_type = unassigned DNA
tacctggatctcagtctcccgaaagaggtgaggag

SEQ ID NO: 41 - DNA, 811 bp, synthetic construct
  misc_feature 1..811: note = Cytomegalovirus enhancer + universal chicken beta-actin gene hybrid promoter
  source 1..811: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccacc

SEQ ID NO: 42 - DNA, 223 bp, synthetic construct
  misc_feature 1..223: note = Bovine-derived growth hormone gene terminator sequence
  source 1..223: mol_type = other DNA
gctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 43 - DNA, 249 bp, synthetic construct
  misc_feature 1..249: note = Human U6 snRNA gene promoter
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 44 - DNA, 35 bp, Homo sapiens
  source 1..35: mol_type = unassigned DNA
ccagaaccggaggacaaagtacaaacggcagaagc

SEQ ID NO: 45 - DNA, 35 bp, Homo sapiens
  source 1..35: mol_type = unassigned DNA
gatgtgatgggagcccttcttcttctgctcggact

SEQ ID NO: 46 - RNA, 35 nt, synthetic construct
  misc_feature 1..35: note = Synthesized
  source 1..35: mol_type = other RNA
ctttcgtccactatgtttcctctggagtgcacacg

SEQ ID NO: 47 - DNA, 56 bp, synthetic construct
  misc_feature 1..56: note = Synthesized
  source 1..56: mol_type = other DNA
tgtgaaagcaggtgatacaaaggagacctcacgtgtgcaaccacctaaaatgaaag

SEQ ID NO: 48 - DNA, 56 bp, synthetic construct
  misc_feature 1..56: note = Synthesized
  source 1..56: mol_type = other DNA
acactttcgtccactatgtttcctctggagtgcacacgttggtggattttactttc

SEQ ID NO: 49 - DNA, 44 bp, synthetic construct
  misc_feature 1..44: note = Synthesized
  misc_difference 4..7: note = n is a, c, g, or t
  source 1..44: mol_type = other DNA
cccnnnncttggtaataatacgactcactatagggagaaaggat

SEQ ID NO: 50 - DNA, 44 bp, synthetic construct
  misc_feature 1..44: note = Synthesized
  source 1..44: mol_type = other DNA
ccataaaggaggtaaataatgaagcagcgtattacagtgacagt

SEQ ID NO: 51 - DNA, 38 bp, synthetic construct
  misc_feature 1..38: note = Synthesized. Includes Nicotiana benthamiana sequences.
  source 1..38: mol_type = other DNA
gtttgggtggtttgtctacagcaaaatatctggcagat

SEQ ID NO: 52 - DNA, 38 bp, synthetic construct
  misc_feature 1..38: note = Synthesized. Includes Nicotiana benthamiana sequences.
  source 1..38: mol_type = other DNA
gtcaaatttgctattggactcttgccagcaatgcttgg

SEQ ID NO: 53 - DNA, 38 bp, synthetic construct
  misc_feature 1..38: note = Synthesized. Includes Solanum lycopersicum sequences.
  source 1..38: mol_type = other DNA
gtctacctggatctcagtctcccgaaagaggtgaggag

SEQ ID NO: 54 - DNA, 56 bp, synthetic construct
  misc_feature 1..56: note = Synthesized. Includes Solanum lycopersicum sequences.
  source 1..56: mol_type = other DNA
ctcaggctcggtctacctggatctcagtctcccgaaagaggtgaggagacttgccc

SEQ ID NO: 55 - DNA, 55 bp, synthetic construct
  misc_feature 1..55: note = Synthesized. Includes Solanum lycopersicum sequences.
  source 1..55: mol_type = other DNA
ctcaggctcggtcacctggatctcagtctcccgaaagaggtgaggagacttgccc

SEQ ID NO: 56 - DNA, 52 bp, synthetic construct
  misc_feature 1..52: note = Synthesized. Includes Solanum lycopersicum sequences.
  source 1..52: mol_type = other DNA
ctcaggctcggtctggatctcagtctcccgaaagaggtgaggagacttgccc

SEQ ID NO: 57 - DNA, 54 bp, synthetic construct
  misc_feature 1..54: note = Synthesized. Includes Solanum lycopersicum sequences.
  source 1..54: mol_type = other DNA
ctcaggctcggtccctggatctcagtctcccgaaagaggtgaggagacttgccc

SEQ ID NO: 58 - DNA, 56 bp, synthetic construct; identical to SEQ ID NO: 54
  misc_feature 1..56: note = Synthesized. Includes Solanum lycopersicum sequences.
  source 1..56: mol_type = other DNA

SEQ ID NO: 59 - DNA, 57 bp, synthetic construct
  misc_feature 1..57: note = Synthesized. Includes Solanum lycopersicum sequences.
  source 1..57: mol_type = other DNA
ctcaggctcggtcttacctggatctcagtctcccgaaagaggtgaggagacttgccc

SEQ ID NO: 60 - DNA, 56 bp, synthetic construct; identical to SEQ ID NO: 54
  misc_feature 1..56: note = Synthesized. Includes Solanum lycopersicum sequences.
  source 1..56: mol_type = other DNA

SEQ ID NO: 61 - DNA, 60 bp, synthetic construct
  misc_feature 1..60: note = Synthesized. Includes Solanum lycopersicum sequences.
  source 1..60: mol_type = other DNA
ctcaggctcggtcaaaatacctggatctcagtctcccgaaagaggtgaggagacttgccc

SEQ ID NO: 62 - DNA, 48 bp, synthetic construct
  misc_feature 1..48: note = Synthesized. Includes Homo sapiens sequences.
  source 1..48: mol_type = other DNA
ggtgtggttccagaaccggaggacaaagtacaaacggcagaagctgga

SEQ ID NO: 63 - DNA, 46 bp, synthetic construct
  misc_feature 1..46: note = Synthesized. Includes Homo sapiens sequences.
  source 1..46: mol_type = other DNA
ggtgtggtcagaaccggaggacaaagtacaaacggcagaagctgga

SEQ ID NO: 64 - DNA, 47 bp, synthetic construct
  misc_feature 1..47: note = Synthesized. Includes Homo sapiens sequences.
  source 1..47: mol_type = other DNA
ggtgtggttcagaaccggaggacaaagtacaaacggcagaagctgga

SEQ ID NO: 65 - DNA, 24 bp, synthetic construct
  misc_feature 1..24: note = Synthesized. Includes Homo sapiens sequences.
  source 1..24: mol_type = other DNA
ggtgtggtaacggcagaagctgga

SEQ ID NO: 66 - DNA, 36 bp, synthetic construct
  misc_feature 1..36: note = Synthesized. Includes Homo sapiens sequences.
  source 1..36: mol_type = other DNA
ggtgtggaggacaaagtacaaacggcagaagctgga

SEQ ID NO: 67 - DNA, 43 bp, synthetic construct
  misc_feature 1..43: note = Synthesized. Includes Homo sapiens sequences.
  source 1..43: mol_type = other DNA
ggtgtgagaaccggaggacaaagtacaaacggcagaagctgga

SEQ ID NO: 68 - DNA, 43 bp, synthetic construct
  misc_feature 1..43: note = Synthesized. Includes Homo sapiens sequences.
  source 1..43: mol_type = other DNA
ggtgtggtaaccggaggacaaagtacaaacggcagaagctgga

SEQ ID NO: 69 - DNA, 48 bp, synthetic construct; identical to SEQ ID NO: 62
  misc_feature 1..48: note = Synthesized. Includes Homo sapiens sequences.
  source 1..48: mol_type = other DNA

SEQ ID NO: 70 - DNA, 46 bp, synthetic construct
  misc_feature 1..46: note = Synthesized. Includes Homo sapiens sequences.
  source 1..46: mol_type = other DNA
ggtgtggttcgaaccggaggacaaagtacaaacggcagaagctgga

SEQ ID NO: 71 - DNA, 47 bp, synthetic construct; identical to SEQ ID NO: 64
  misc_feature 1..47: note = Synthesized. Includes Homo sapiens sequences.
  source 1..47: mol_type = other DNA

SEQ ID NO: 72 - DNA, 24 bp, synthetic construct
  misc_feature 1..24: note = Synthesized. Includes Homo sapiens sequences.
  source 1..24: mol_type = other DNA
ggtgtggtaacggcagaagctgga

SEQ ID NO: 73 - DNA, 42 bp, synthetic construct
  misc_feature 1..42: note = Synthesized. Includes Homo sapiens sequences.
  source 1..42: mol_type = other DNA
ggtgtggaaccggaggacaaagtacaaacggcagaagctgga

SEQ ID NO: 74 - DNA, 48 bp, synthetic construct
  misc_feature 1..48: note = Synthesized. Includes Homo sapiens sequences.
  source 1..48: mol_type = other DNA
ctgagtccgagcagaagaagaagggctcccatcacatcaaccggtggc

SEQ ID NO: 75 - DNA, 47 bp, synthetic construct
  misc_feature 1..47: note = Synthesized. Includes Homo sapiens sequences.
  source 1..47: mol_type = other DNA
ctgagtccgagcagaagaagaagggctcccatcacataaccggtggc

SEQ ID NO: 76 - DNA, 46 bp, synthetic construct
  misc_feature 1..46: note = Synthesized. Includes Homo sapiens sequences.
  source 1..46: mol_type = other DNA
ctgagtccgagcagaagaagaagggctcccatcacaaaccggtggc

SEQ ID NO: 77 - DNA, 38 bp, synthetic construct
  misc_feature 1..38: note = Synthesized. Includes Homo sapiens sequences.
  source 1..38: mol_type = other DNA
ctgagtccgagcagaagaagaagggctccaccggtggc

SEQ ID NO: 78 - DNA, 45 bp, synthetic construct
  misc_feature 1..45: note = Synthesized. Includes Homo sapiens sequences.
  source 1..45: mol_type = other DNA
ctgagtccgagcagaagaagaagggctcccatcacatccggtggc

SEQ ID NO: 79 - DNA, 45 bp, synthetic construct
  misc_feature 1..45: note = Synthesized. Includes Homo sapiens sequences.
  source 1..45: mol_type = other DNA
ctgagtccgagcagaagaagaagggctcccatcacattcggtggc

SEQ ID NO: 80 - DNA, 48 bp, synthetic construct; identical to SEQ ID NO: 74
  misc_feature 1..48: note = Synthesized. Includes Homo sapiens sequences.
  source 1..48: mol_type = other DNA

SEQ ID NO: 81 - DNA, 47 bp, synthetic construct; identical to SEQ ID NO: 75
  misc_feature 1..47: note = Synthesized. Includes Homo sapiens sequences.
  source 1..47: mol_type = other DNA

SEQ ID NO: 82 - DNA, 45 bp, synthetic construct
  misc_feature 1..45: note = Synthesized. Includes Homo sapiens sequences.
  source 1..45: mol_type = other DNA
ctgagtccgagcagaagaagaagggctcccatcacaaccggtggc

SEQ ID NO: 83 - DNA, 27 bp, synthetic construct
  misc_feature 1..27: note = Synthesized. Includes Homo sapiens sequences.
  source 1..27: mol_type = other DNA
ctgagtccgagcagaagaaccggtggc

SEQ ID NO: 84 - DNA, 59 bp, synthetic construct
  misc_feature 1..59: note = Synthesized. Includes Homo sapiens sequences.
  source 1..59: mol_type = other DNA
agcgggactccgaaaggagggagacgagctcatccctcgggccttacttgcagctcgca

SEQ ID NO: 85 - DNA, 86 bp, synthetic construct
  misc_feature 1..86: note = Synthesized. Includes Homo sapiens sequences.
  source 1..86: mol_type = other DNA
ctgagtccgagcagaagaagcgggactccgaaaggagggagacgagctcatccctcgggccttacttgcagctcgcaaccggtggc

(19) United States
(12) Patent Application Publication
(10) Pub. No.: US 2024/0392288 A1
(43) Pub. Date: Nov. 28, 2024

(54) TARGET SEQUENCE SPECIFIC ALTERATION TECHNOLOGY USING NUCLEOTIDE TARGET RECOGNITION

(71) Applicant: Tokushima University, Tokushima (JP)

(72) Inventors: Keishi Osakabe, Tokushima (JP); Yuriko Osakabe, Tokushima (JP)

(21) Appl. No.: 18/743,834

(22) Filed: Jun. 14, 2024

Related U.S. Application Data
(63) Continuation of application No. 16/640,521, filed on Feb. 20, 2020, now Pat. No. 12,012,596.

(30) Foreign Application Priority Data
Aug. 21, 2017 (JP) 2017-158876
Dec. 8, 2017 (JP) 2017-236518

(51) Int. Cl. C12N 15/11; C12N 9/22; C12N 15/10

(52) U.S. Cl. CPC C12N 15/111; C12N 9/22; C12N 15/102; C12N 2310/20

(57) ABSTRACT

Provided is a method for targeting a target nucleotide sequence. The method includes introducing, into a cell: (i) CRISPR type I-D related proteins Cas5d, Cas6d, and Cas7d, or nucleic acids encoding these proteins; and (ii) a guide RNA which includes a sequence complementary to said target nucleotide sequence and common repetitive sequences derived from CRISPR gene locus before and after said complementary sequence, or a DNA encoding said guide RNA.

### CROSS REFERENCE TO RELATED APPLICATION

The present application is a Continuation of U.S. patent application No. 16/640,521, filed on Feb. 20, 2020, which claims priority to International Patent Application No. PCT/JP2018/030607 filed Aug. 20, 2018, which claims priority to Japanese Patent Application No 2017-158876, filed Aug. 21, 2017 and Japanese Patent Application No. 2017-236518, filed Dec. 8, 2017. The entire contents of these applications are incorporated herein for all purposes by this reference.

### REFERENCE TO A SEQUENCE LISTING

This application contains a Sequence Listing which has been submitted electronically in xml format and is hereby incorporated by reference in its entirety. Said xml copy, created on Aug. 13, 2024, is named SeqList2-348819-00221.xml and is 131,131 bytes in size.

### TECHNICAL FIELD

The present invention relates to a method for targeting a target nucleotide sequence, a method for specifically altering a target nucleotide sequence, and a method for suppressing the expression of a target gene, wherein the nucleotide target recognition of CRISPR (Clustered Regularly Interspaced Short Palindromic Repeats) type I-D system is utilized, and a complex comprising Cas (CRISPR-associated) proteins and a guide RNA used in the methods.

### Background Art

Bacteria and archaea have CRISPR systems as an adaptive immune system against viruses and heterologous foreign plasmids. The CRISPR system uses a low molecular RNA (referred to as a guide RNA or gRNA) that is complementary to an invading DNA sequence to promote targeting and degradation of the target foreign DNA. At this time, Cas protein that binds to the gRNA to form a complex is required. The CRISPR system includes type I, type II, type III, and Type V systems. In any system, the Cas protein-gRNA complex acts on the target sequence to cause interference with viruses and foreign plasmids. In type II and type V systems, the mechanism of interference involves DNA double-strand breaks on a target DNA by an integrated protein having a protein domain that retains gRNA-binding and a RuvC-Like DNA-cleaving protein domain. For the type III system, it has been demonstrated in vitro and in vivo that the interference is caused by cleavage of a target RNA sequence by a complex of five to eight Cas proteins and a gRNA, unlike the type II system.

In recent years, genome editing techniques using the CRISPR type II and type V systems have been developed, wherein Cas9 and Cpf1 are utilized as the Cas protein. Cas9 and Cpf1 require a sequence consisting of about 2 to 5 nucleotides in the vicinity of a target sequence, which is called a protospacer adjacent motif (PAM) sequence, in order to recognize the target DNA. It has been demonstrated in vitro and in vivo that a Cas9-gRNA complex and a Opf1-gRNA complex are sequence-specific RNA-guided endonucleases that cause DNA double-strand breaks at target sites near PAM sequences.

On the other hand, regarding the CRISPR type I system, a plurality of subtypes have been identified in genomic sequences from various bacteria, and the subtypes have been named type I-A, I-B, I-C, I-D, I-E, I-F, and I-U. Among these subtypes, the type I-E system derived from Escherichia coli has been most studied, and it has been demonstrated that a complex consisting of six Cas proteins (Cas3, Cse1, Cse2, Cas7, Cas5, Cas6e) and a gRNA promotes degradation of a target DNA sequence. For the other subtypes excluding a subtype (type I-C), however, Cas protein components, gRNA sequences, PAM sequences that determine target DNAs, etc. required for the interference effect are hardly elucidated. In addition, as techniques using Cas proteins derived from the CRISPR type I system, a method for suppressing the expression of a target gene which comprises using recombinant nucleic acid molecules encoding Cas proteins derived from the CRISPR type I system (Patent Literature 1), and a method for altering a target nucleic acid which comprises using a complex of Cas proteins derived from the CRISPR type I system and other proteins (Patent Literature 2 and Patent Literature 3) have been reported. However, a technique for cleaving and altering a double strand of a target DNA molecule by RNA-guided endonuclease derived from the CRISPR type I system has never been reported.

### CITATION LIST

### Patent Literature

Patent Literature 1: 02015/155686

Patent Literature 2: JP-A 2015-503535

Patent Literature 3: WO2017/043573

### SUMMARY OF INVENTION

### Technical Problems

In the conventional CRISPR type II and type V systems, an RNA molecule to be utilized for targeting is limited to an RNA molecule of about 20 nucleotides preceding or following a PAM sequence of about 2 to 5 nucleotides which determines target specificity. Thus the conventional CRISPR type II and type V systems have problems that there are loci in which a target cannot be designed and that similar sequences may be cleaved. The development of a novel targeting system and a novel RNA-guided endonuclease that does not have the problems is desired.

### Solution to Problems

In order to solve the above problems, the present inventors intensively studied. As a result, surprisingly, a novel targeting system and a novel RNA-guided endonuclease that target a longer sequence than the target sequence of the CRISPR type II or type V RNA-guided endonucleases conventionally used in genomes editing technology were found from CRISPR type I-D, and then it was found that the novel targeting system and RNA-guided endonuclease can be used in genome editing techniques for allowing alteration on a target nucleotide sequence. Thus the present invention was completed.

That is, the present invention provides:
[1] A method for targeting a target nucleotide sequence, the method comprising introducing into a cell:
(i) CRISPR type I-D associated proteins Cas5d, Cas6d and Cas7d, or nucleic acids encoding the proteins, and
(ii) a guide RNA comprising a sequence complementary to the target nucleotide sequence, and common repetitive sequences derived from a CRISPR locus, preceding and following the complementary sequence, or a DNA encoding the guide RNA;
[2] A method for altering a target nucleotide sequence, the method comprising introducing into a cell:
(i) CRISPR type I-D associated proteins Cas3d, Cas5d, Cas6d, Cas7d and Cas10d, or nucleic acids encoding the proteins, and
(ii) a guide RNA comprising a sequence complementary to the target nucleotide sequence, and common repetitive sequences derived from a CRISPR locus, preceding and following the complementary sequence, or DNA encoding the guide RNA;
[3] A method for suppressing the expression of a target gene, the method comprising introducing into a cell:
(i) CRISPR type I-D associated proteins Cas5d, Cas6d and Cas7d, or nucleic acids encoding the proteins, and
(ii) a guide RNA comprising a sequence complementary to at least a part of the target gene sequence, and common repetitive sequences derived from a CRISPR locus, preceding and following the complementary sequence, or a DNA encoding the guide RNA;
[4] The method according to any one of [1] to [3], wherein the guide RNA comprises a sequence consisting of 20 to 50 nucleotides which is complementary to the target nucleotide sequence;
[5] The method according to [2] or [4], further comprising introducing a donor polynucleotide into the cell;
[6] The method according to any one of [2], [4] and [5], wherein the alteration is nucleotide deletion, insertion, or substitution;
[7] The method according to any one of [1] to [6], wherein the Cas5d recognizes 5′-GTH-3′ (H=A, C, or T) as a protospacer adjacent motif (PAM) sequence;
[8] A complex comprising:
(i) CRISPR type I-D associated proteins Cas5d, Cas6d and Cas7d, and
(ii) a guide RNA comprising a sequence complementary to a target nucleotide sequence, and common repetitive sequences derived from a CRISPR Locus, preceding and following the complementary sequence;

[9] The complex according to [8], further comprising Cas3d and Cas10d;
[10] The complex according to [8] or [9], wherein the guide RNA comprises a sequence consisting of 20 to 50 nucleotides which is complementary to the target nucleotide sequence;
[11] An expression vector comprising:
(i) nucleic acids encoding CRISPR type I-D associated proteins Cas5d, Cas6d and Cas7d, and
(ii) a DNA encoding a guide RNA comprising a sequence complementary to a target nucleotide sequence, and common repetitive sequences derived from a CRISPR locus, preceding and following the complementary sequence;
[12] The expression vector according to [11], further comprising nucleic acids encoding Cas3d and Cas10d;
[13] A DNA molecule encoding the complex according to any one of [8] to [10];
[14] Use of
(i) CRISPR type I-D associated proteins Cas5d, Cas6d and Cas7d, or nucleic acids encoding the proteins, and
(ii) a guide RNA comprising a sequence complementary to a target nucleotide sequence, and common repetitive sequences derived from a CRISPR locus, preceding and following the complementary sequence, or a DNA encoding the guide RNA,
   for targeting the target nucleotide sequence;
[15] Use of
(i) CRISPR type I-D associated proteins Cas3d, Cas5d, Cas6d, Cas7d and Cas10d, or nucleic acids encoding the proteins, and
(ii) a guide RNA comprising a sequence complementary to a target nucleotide sequence, and common repetitive sequences derived from a CRISPR locus, preceding and following the complementary sequence, or a DNA encoding the guide RNA, for altering the target nucleotide sequence;
[16] Use of
   (i) CRISPR type I-D associated proteins Cas5d, Cas6d and Cas7d, or nucleic acids encoding the proteins, and
(ii) a guide RNA comprising a sequence complementary to at least a part of a target gene sequence, and common repetitive sequences derived from a CRISPR locus, preceding and following the complementary sequence, ox a DNA encoding the guide RNA,
   for suppressing the expression of the target gene;
[17] Use according to any one of [14] to [16], wherein the guide RNA comprises a sequence consisting of 20 to 50 nucleotides complementary to the target nucleotide sequence;
[18] Use according to [15] or [17], wherein the alteration is nucleotide deletion, insertion, or substitution;
[19] Use according to any one of to [18], wherein the Cas5d recognizes 5′-GTH-3′ (H=A, C, or T) as a protospacer adjacent motif (PAM) sequence;
[20] Use of a complex comprising:

(i) CRISPR type I-D associated proteins Cas5d, Cas6d and Cas7d, and
(ii) a guide RNA comprising a sequence complementary to a target nucleotide sequence, and common repetitive sequences derived from a CRISPR locus, preceding and following the complementary sequence, for targeting the target nucleotide sequence;
[21] Use of a complex comprising:
(i) CRISPR type I-D associated proteins Cas3d, Cas5d, Cas6d, Cas7d and Cas10d and
(ii) a guide RNA comprising a sequence complementary to a target nucleotide sequence, and common repetitive sequences derived from a CRISPR locus, preceding and following the complementary sequence, for altering the target nucleotide sequence;
[22] Use of the complex comprising:
(i) CRISPR type I-D associated proteins Cas5d, Cas6d and Cas7d, and
(ii) a guide RNA comprising a sequence complementary to at least a part of a target gene sequence, and common repetitive sequences derived from a CRISPR locus, preceding and following the complementary sequence, for suppressing the expression of the target gene; and
[23] Use according to any one of [20] to [22], wherein the guide RNA comprises a sequence consisting of 20 to 50 nucleotides complementary to the target nucleotide sequence.

### Effects of the Invention

The PAM sequences of the CRISPR type I-D (hereinafter, also referred to as “TiD”) system are different from the PAM sequences of the CRISPR type II system and the type V system. Therefore, according to the present invention, the use of the Cas proteins of CRISPR type I-D makes it possible to target loci that cannot be targeted by the conventional genome editing techniques using CRISPR type II or type V RNA-guided endonucleases. Furthermore, the PAM sequences of the CRISPR type I-D-derived RNA-guided endonuclease of the present invention are more frequently found on the genomic sequences of some organisms than the PAM sequences of CRISPR type II and type V. Therefore, according to the present invention, it is possible to target a larger number of gene sequences than the conventional genome editing techniques utilizing the CRISPR type II and type V systems. Furthermore, the present inventors found that a gRNA in the CRISPR type I-D system can target a target sequence having a length of 30 nucleotides or more. On the other hand, a gRNA in the CRISPR type II or type V system can target a sequence having a length of about 20 nucleotides. Thus, the CRISPR type I-D system of the present invention shows more stable binding properties and target specificity than the conventional techniques.

Thus, according to the present invention, it is possible to realize the generation of mutant alleles, control of gene expression by transcriptional activation and inactivation, and epigenomic alteration by targeting of a DNA-modifying/histone-modifying protein domain, on gene regions that cannot be targeted by the conventional techniques.

### BRIEF DESCRIPTION OF DRAWINGS

FIG. 1 outlines the component of the CRISPR type I-D system of the present invention and the targeting and cleavage modes on a target sequence.

FIG. 2 shows the TiD expression vector for E. coli genome editing: a) the structure of pEcTiD2 plasmid; b) the structure of pEcTiD3 plasmid, Pro: J23108 synthetic promoter, t1: terminator sequence STOP767, RES: ribosome binding sequence, t2: terminator sequence STOP768 (1), t3: terminator sequence TOP768 (2), t7: 17 terminator sequence, 7d: Cas7d derived from Microcystis aeruginosa (hereinafter, abbreviated as “Ma”), 6d: MaCas6d, 5d: MaCas5d, 3d: MaCas3d, 10d: MaCas10d, 17 pro: 17 promoter, crRNA: TiD-derived CRISPR repeat sequence, Om: chloramphenicol resistance gene, p15A ori: p15A plasmid-derived replication origin.

FIG. 3 shows the structures of pMW_ccdB and pMW_ccdB-PAM Library plasmids: a) the structure of pMW_ccdB, t2: rrnB2 terminator sequence, t1: rrnB1 terminator sequence, PAM: protospacer adjacent motif sequence, T7 pro: T7 promoter, ccdB: ccdB gene, Km: kanamycin resistance gene, pSC101 ori: pSC101 plasmid-derived replication origin; b) the target sequence of pMW ccdB-PAM plasmid library, wherein random 4 nucleotides are inserted into the NNNN site to obtain a PAM sequence screening library plasmid, a boxed region indicates the T7 promoter, the underlined sequence indicates the TiD target sequence, and capital letters indicate the ccdB locus,

FIG. 4 shows the TiD expression vector for plant genome editing: a) the structure of pEgPTiD1 plasmid; b) the structure of crRNA expression cassette for plants; c) the structure of pEgPTiD2 plasmid, RB: right border sequence, LB: left border sequence, 2×358: 2× cauliflower mosaic virus 35S gene promoter and translation enhancer Ω sequence, 3d: MaCas3d with a sequence encoding 2×NLS (nuclear localizing signal), 10d: MaCas10d with 2×NLS, 7d: MaCas7d with 2×NLS, 6d: MaCas6d with 2×NLS, 5d: MaCas5d with 2×NLS, 2A (1)-(4): self-cleaving peptide 2A sequences (1)-(4), Ter: Arabidopsis heat shock protein 18.2 kDa gene terminator, Km: kanamycin resistance gene expression cassette, U6-26: Arabidopsis U6 snRNA-26 gene promoter, CrRNA: TiD locus-derived CRISPR repeat sequences.

FIG. 5-1 shows the mutagenesis of the tobacco PDS gene using pEgPTiD2-pds: a) target sequences on the tobacco PDS gene, wherein target sequence 1 was selected from the third exon and target sequence 2 was selected from the sixth exon, boxed parts in the target sequences shown in the lower panel indicates the PAM sequences and the underlined parts indicate the target sequences; b) introduction of pEgPTiD2-pds and a GFP expression binary vector by agroinfiltration, wherein agrobacteria carrying pEgTiD2-pds (1) or pEgPTiD-pds (2) and Agrobacteria carrying a GFP expression binary plasmid were infected by agroinfiltration, and leaf discs in which the GFP expression was observed were excised and used for the analysis of the FDS mutation introduction.

FIG. 5-2 shows the site-directed mutagenesis of the tobacco PDS gene using pEgPTiD2-pds: c) analysis of the PDS mutation introduction by the Cel-1 assay, wherein the genomic DNA was prepared from the leaf discs in which the GFP expression was observed in FIG. 5-b), and the presence or absence of mutations was analyzed by the Cel-1 assay. Triangle marks indicate mutated PDS gene fragments that were cleaved by the Cel-1 nuclease.

FIG. 6 shows the mutagenesis of the tomato IAA9 gene using pEgPTiD2-iaa9; a) a target sequence on the tomato IAA9 gene, wherein target sequence 1 was selected from the second exon, a boxed part in the target sequence shown in the lower panel indicates the PAM sequence, and the underlined part indicates the target sequence; b) pEgPTiD2-iaa9 was introduced into a tomato leaf disc by Agrobacterium method to obtain transformed callus cells; c) mutation analysis by PCR-RFLP, wherein a region containing the IAA9 target sequence was amplified by PCR from a genomic DNA that was prepared from the transformed callus cells into which pEgPTiD2-iaa9 was introduced, and the mutation analysis was performed by PCR-RFLP using AccI. Open triangles indicate wild type-derived AccI cleavage fragments, and a triangle above the open triangles indicates a mutated fragment that does not undergo AccI cleavage.

FIG. 7 shows mutation analysis by sequencing in pEcTiD2-iaa9 introduced calluses. The top sequence shows the wild type IA49 sequence, and an underlined part indicates the target sequence. Boxed sequences indicate the PAM sequences. Sites, where mutations occurred, are shown by insertion symbols or hyphens. The hyphen indicates nucleotide deletion.

FIG. 8 shows mutation analysis in a pEcTiD2-iaa9-introduced regenerated plant: a) mutation analysis by PCR-RFLP, wherein open triangles indicate wild type-derived AccI cleavage fragments, and a triangle above the open triangles indicates mutated fragments that do not undergo AccI cleavage; and b) a photograph of a mutation-introduced tomato plant that shows abnormal morphology of true leaves as a result of IAA9 gene disruption.

FIG. 9 shows an experimental scheme of genome editing using the HEK293 cell line.

FIG. 10 shows results of mutation analysis by the heteroduplex mobility analysis. A fragment considered to be derived from a mutated sequence was detected (black key symbol) from the genome of a call into which a crRNA containing the sequence of target 1 on the EMX1 gene and the TiD genes were introduced.

FIG. 11 shows results of mutation analysis by the heteroduplex mobility analysis. A fragment considered to be derived from a mutated sequence was detected (black key symbol) from the genome of a cell into which a crRNA containing the sequence of target 2 on the EMX1 gene and the TiD genes were introduced.

FIG. 12 shows the sequencing analysis of mutated sequences. White letters in black backgrounds indicate PAM (protospacer adjacent sequence) recognized by TiD. Boxed sequences indicate target sequences. Hyphens (-) indicate nucleotide deletion. A black bold lower case alphabetic character indicates nucleotide insertion. On the right side of each sequence, somatic mutation efficiency (the number of clones in which a mutated sequence was observed/the total number of analyzed clones) is shown.

FIG. 13 shows the sequencing analysis of mutated sequences. White letters in black backgrounds indicate PAM (protospacer adjacent sequence) recognized by TiD. Boxed sequences indicate target sequences. Hyphens (-) indicate nucleotide deletion. Black bold lower case alphabetic characters indicate nucleotide insertion. On the right side of each sequence, somatic mutation efficiency (the number of clones in which a mutated sequence was observed/the total number of analyzed clones) is shown.

### MODE FOR CARRYING OUT THE INVENTION

The present invention provides a genome editing technique utilizing the CRISPR type I-D system. Specifically, among CRISPR type I-D Cas proteins, Cas3d, Cas5d, Cas6d, Cas7d and Cas10d are used in the present invention. In the present invention, the CRISPR type I-D system was found to comprise a target recognition module comprising Cas5d, Cas6d and Cas7d and a polynucleotide cleavage module comprising Cas3d and Cas10d.

Specifically, the action principle of the present invention is as follows.

A complex comprising:
1) a gRNA which is necessary for targeting of a target nucleotide sequence (hereinafter also referred to as “targeting”), comprising a sequence complementary to the target nucleotide sequence and a common repetitive sequence present in a CRISPR type I-D locus,
2) Cas5d which recognizes a PAM sequence present in the vicinity of the target nucleotide sequence,
3) Cas7which binds to the gRNA of 1) and is necessary for targeting of the target nucleotide sequence, and
4) Cas6d which performs processing of the gRNA of 1), and
5) a complex comprising Cas10d which interacts with the complex comprising 1) to 4) and performs remodeling of the target nucleotide sequence and Cas3d which performs degradation of a polynucleotide are provided to a call and in the call,
6) targeting of the target nucleotide sequence by the complex comprising 1) to 4), that is,
7) targeting of the target nucleotide sequence by a complex comprising a mature gRNA that is obtained by processing of the gRNA of. 1) by Cas6d of 4), and 2) and 3)
   is performed, and
8) a polynucleotide on the target nucleotide sequence is cleaved by the complex of 5).

Therefore, the present invention provides a method for targeting a target nucleotide sequence (hereinafter also referred to as “the target sequence-targeting method of the present invention”), a method for altering a target nucleotide sequence (hereinafter referred to as “the target sequence-altering method of the present invention”), and a method for suppressing the expression of a target gene (hereinafter also referred to as “the target gene expression-suppressing method of the present invention”), wherein the CRISPR type I-D system is utilized in the methods. Furthermore, the present invention provides a complex comprising CRISPR type I-D-associated Cas proteins and a gRNA (hereinafter also referred to as “the complex of the present invention”), and a vector comprising a nucleic acid molecule encoding the complex, which are used in the above-mentioned methods of the present invention.

### (1) Cell

In the present invention, the cell may be either a prokaryotic cell or a eukaryotic cell, and is not particularly limited. Examples of the cell include bacteria, archaea, yeast, plant cells, insect cells, and animal cells (e.g., human cells, non-human cells, non-mammalian vertebrate cells, invertebrate cells, etc.).

### (2) RNA-Guided Endonuclease and Cas Protein

In the present invention, the “RNA-guided endonuclease” means an endonuclease comprising at least one nuclease domain and at least one domain that binds to a gRNA, which is guided to a target nucleotide sequence (or a target nucleotide site) by the gRNA. The RNA-guided endonuclease used in the present invention is an RNA-guided endonuclease derived from CRISPR type I-D, and comprises CRISPR type I-D-associated proteins Cas3d, Cas5d, Cas6d, Cas7d and Cas10d. In the present invention, it was found that Cas5d, Cased and Cas7d constitute a “target recognition module” that contributes to target recognition, and Cas3d and Cas10d constitute a “polynucleotide cleavage module” that contributes to cleavage of a polynucleotide. Specifically, the RNA-guided endonuclease used in the present invention comprises the target recognition module comprising Cas5d, Cas6d and Cas7d and the polynucleotide cleavage module comprising Cas3d and Cas10d.

The Cas3d, Cas5d, Cas6d, Cas7d and Cas10d used in the present invention may be derived from any bacterium or archaeum. Examples of the bacterium and the archaeum include Microcystis aeruginosa, Acetohalobium arabaticum, Ammonifex degensii, Anabaena cylindrica, Anabaena variabilis, Caldicellulosiruptor lactoaceticus, Caldilinea aerophila, Clostridium algicarnis, Crinaliun epipsammaum, Cyanothece Sp., Cylindrospermum stagnale, Baloquadratum walsbyi, Halorubrum lacusprofundi, Methanocaldococcus vulcanius, Methanospirillum hungatei, Natrialba asiatica, Natrona pharaonis, Nostoc punctiforme, Phormidesmis priestleyi, Oscillatoria acuminata, Picrophilus torridus, Spirochaeta thermophila, Stanieria cyanosphaera, Sulfolobus acidocaldarius, Sulfolobus islandicus, Synechocystis Sp., Thermacetogenium phaeum, Thermofilum pendens, etc. The amino acid sequence and nucleotide sequence information of the Cas proteins are available from public database, for example, NCBI GenBank. In addition, the sequences from novel microbial species can be also obtained from microbial genome data obtained by metagenomic analysis or the like by using the BLAST program. Nucleic acids encoding the Cas proteins may be constructed, for example, by chemical synthesis or the like after selecting optimum codons for translation in a host cell into which the nucleic acids are introduced on the basis of the amino acid sequence information. Use of codons that are frequently used in a host cell makes it possible to increase the expression level of protein. For example, the Cas proteins may be chemically synthesized based on the amino acid sequence information, or produced in a cell by introducing nucleic acids encoding the Cas proteins into the cell via an appropriate vector or the like. Each Cas protein of Cas3d, Cas5d, Cas6d, Cas7d and Cas10d may be a mutant-type Cas protein as long as it retains the function of each Cas protein as described in the action principle of the present invention.

### (3) Guide RNA

In the present invention, the guide RNA (gRNA) is a molecule that forms a complex with the target recognition module (Cas5d, Cas6d and Cas7d) to target a target nucleotide sequence together with these Cas proteins. In the present invention, the gRNA binds to Cas7d of the target recognition module. In the present invention, the gRNA binds to a complex comprising Cas5d, Cas6d and Cas7d to guide the complex to the target nucleotide sequence. For example, the gRNA binds to the target. recognition module of the RNA-guided endonuclease to guide the RNA-guided endonuclease to the target nucleotide sequence. When the target recognition module is present as a part of a fusion protein other than the RNA-guided endonuclease, the gRNA binds to the target recognition module to guide the fusion protein to the target nucleotide sequence.

The gRNA comprises a sequence complementary to a target sequence so that a base pair may be formed between the gRNA and the target nucleotide sequence, and common repetitive sequences derived from a CRISPR type I-D locus preceding and following (at the S′-end side and the 3′mend side of) the complementary sequence. The common repetitive sequence parts of gRNA may have at least one hairpin structure. For example, the common repetitive sequence part placed at the 5′-end side of the sequence complementary to a target nucleotide sequence may have a hairpin structure, and the common repetitive sequence part placed at the 3′-end side of the sequence complementary to a target nucleotide sequence may be single-stranded. It the present invention, the gRNA preferably has a hairpin structure.

The common repetitive sequence derived from a CRISPR type I-D locus can be found from a gRNA gene sequence region adjacent to a type I-D gene group by using a ta at search program. The nucleotide length of the common repetitive sequence contained in the gRNA is not particularly limited as long as the gRNA interacts with the target recognition module to target a target nucleotide sequence. For example, each of the common repetitive sequences preceding and following the sequence complementary to a target nucleotide sequence may have a length of about 10 to 70 nucleotides, for example, a length of 30 to 50 nucleotides.

The gRNA can contain a sequence consisting of about 10 to 70 nucleotides, which is complementary to a target nucleotide sequence. The sequence complementary to a target nucleotide sequence contained in the gRNA is preferably a sequence consisting of 20 to 50 nucleotides, more preferably a sequence consisting of 25 to 45 nucleotides, more preferably a sequence consisting of 30 to 40 nucleotides, or even more preferably a sequence consisting of 32 to 37 nucleotides, for example, a sequence consisting of 32 nucleotides, 33 nucleotides, 34 nucleotides, 35 nucleotides, 36 nucleotides, or 37 nucleotides. The sequence specificity of target recognition by the gRNA is more greatly increased as the target sequence that can be targeted is longer. In addition, the Tm value of a base pair formed between the gRNA and the target sequence is higher and thus the stability of target recognition is more greatly increased as the target sequence that can be targeted is longer. Since the length of a sequence that can be targeted by a gRNA for RNA-guided endonucleases (e.g., Cas9 and Cpf1) used in the conventional genome editing techniques is about 20 to 24 nucleotide length, the present invention is excellent in the sequence specificity and the stability as compared with the conventional method is.

### (4) Target Nucleotide Sequence

In the present invention, the target nucleotide sequence (also referred to as “the target sequence”, as used herein) is any nucleic acid sequence, and is not particularly limited as long as it is a sequence located in the vicinity of a protospacer proximity motif (PAM). The target nucleotide sequence may be a double-stranded DNA sequence, a single-stranded DNA sequence, or an RNA sequence. Examples of DNA include eukaryotic nuclear genomic DNA, mitochondrial DNA, plastid DNA, prokaryotic genomic DNA, phage DNA, and plasmid DNA. In the present invention, the target nucleotide sequence is preferably a double-stranded DNA on the genome. As used herein, the phrase “in the vicinity of” includes both being adjacent to a place and being close to a place. As used herein, the “vicinity” includes both adjacency and neighborhood.

The PAM sequences used for target recognition of CRISPR systems vary depending on the types of CRISPR systems. In the present invention, it was found that the PAM sequence utilized by the CRISPR type I-D system is 5′-GTH-3′ (H=A, C or T) (Example 1). Preferably, a sequence located in the vicinity of the 3′-downstream side of the PAM sequence is selected as the target nucleotide sequence. For example, the target nucleotide sequence may be a sequence located in the vicinity of the PAM sequence and present in an intron, a coding region, a non-coding region, or a control region of a target gene. The target gene may be any gene and optionally selected.

The PAM sequences for Cas9 and Cpf1 used in the conventional genome editing techniques are 5′-NGG-3′ (N=A, C, G or T) and 5′-TTTV-3′ (V=A, C or G), respectively. The appearance frequency of the FAM sequence for TiD (i.e., the number of candidate targets of the CRISPR system) was compared with the appearance frequencies of the PAM sequences for Cas9and Opf1 in the genome sequences of higher plants. As a result, it was found that the appearance frequency of the PAM sequence for TiD is the highest, and TiD has a larger number of targets than the conventional genome editing techniques using Cas9 and Cpf1 (Table 1).

TABLE 1
Comparison of PAM sequence number (=number of candidate
targets) in genome sequences of higher plants

| Plant species | TiD | Cas9 | Cpf1 |
|---|---|---|---|
| Tomato | 5.99 × 10⁷ | 5.10 × 10⁷ | 3.97 × 10⁷ |
| Strawberry | 1.70 × 10⁷ | 1.68 × 10⁷ | 0.99 × 10⁷ |
| Potato | 5.45 × 10⁷ | 4.99 × 10⁷ | 3.71 × 10⁷ |
| Soybean | 7.20 × 10⁷ | 6.96 × 10⁷ | 5.38 × 10⁷ |
| Hot pepper | 21.58 × 10⁷ | 20.19 × 10⁷ | 13.60 × 10⁷ |
| Poplar | 2.98 × 10⁷ | 2.77 × 10⁷ | 2.39 × 10⁷ |
| Cabbage | 3.67 × 10⁷ | 3.19 × 10⁷ | 2.42 × 10⁷ |
| Beet | 4.11 × 10⁷ | 4.06 × 10⁷ | 2.70 × 10⁷ |
| Bur clover | 3.08 × 10⁷ | 2.48 × 10⁷ | 2.31 × 10⁷ |

### (5) The Method of Targeting a Target Sequence of the Present Invention

The method of targeting a target sequence of the present invention is characterized by introducing the target recognition module (Cas5d, Cas6d and Cas7d) and the gRNA into the cell. Specifically, the target sequence-targeting method of the present invention is characterized by introducing into the cell (i) Cas5d, Cas6d and Cas7d, or nucleic acids encoding these proteins, and (ii) the gRNA or a DNA encoding the gRNA. The target sequence-targeting method of the present invention may be performed in vitro or in vivo.

In the method of targeting a target sequence of the present invention, the target recognition module may be introduced into the cell as an isolated complex comprising Cas5d, Cas6d and Cas7d, or each of Cas5d, Cas6d and Cas7 d may be introduced into the cell as an isolated single protein. In the target sequence-targeting method of the present invention, the target recognition module may be also introduced into the cell as nucleic acids encoding Cas proteins Cas5d, Cas6d and Cas7d. Examples of the nucleic acid include RNA such as miRNA and DNA.

DNAs encoding the Cas proteins may be contained in, for example, a vector. The DNA sequence is preferably operably linked to a regulatory sequence such as a promoter or terminator. When the cell into which the target recognition module is introduced is a eukaryotic cell, a nuclear localizing signal sequence is preferably added to the DNA encoding the Cas protein. Two or more or all of the DNAs encoding the Cas proteins Cas5d, Cas6d and Cas7d may be contained in a single vector or may be contained in separate vectors. The number of vectors and the kinds and combinations of Cas proteins encoded by the DNAs to be incorporated into each vector are not limited. When two or more DNAs encoding the Cas proteins are contained in a single vector, the DNA sequences may be linked to each other, for example via a sequence encoding a self-cleaving peptide, so as to be polycistronically expressed. The two or more DNAs encoding the Cas proteins may be linked in any order.

The gRNA may be introduced into the cell as an RNA or as a DNA encoding the gRNA. The DNA encoding the gRNA may be contained, for example, in a vector. The DNA sequence is preferably operably linked to a regulatory sequence such as a promoter or a terminator.

The DNAs encoding the Cas proteins and the DNA encoding the gRNA may be contained in the same vector or may be contained in separate vectors. For example, one or more or all of the DNAs encoding Cas5d, Cas6d and Cas7d, and the DNA encoding the gRNA may be contained in a single vector.

The regulatory sequence such as a promoter or a terminator and the nuclear localizing signal sequence are known in the art and can be appropriately selected depending on organism species in which the cell into which the target recognition module and the gRNA are introduced is derived from the organism species. The vector used for introduction may be appropriately selected depending on organism species in which the cell into which the vector is introduced is derived from the organism species, and is not particularly limited. Examples of the vector include plasmid vectors, virus vectors, phagemids, cosmids, artificial/mini-chromosomes, and transposons.

The introduction of the target recognition module and the gRNA into the cell can be performed by various means known in the art. Examples of such means include transfection, e.g., calcium phosphate-mediated transfection, electroporation, liposome transfection, etc., virus transduction, lipofection, gene gun, microinjection, Agrobacterium method, Agroinfiltration, and a PEG-calcium method.

The target recognition module and the gRNA may be introduced into the cell simultaneously or sequentially. Cas5d, Cas6d and Cas7d constituting the target recognition module, or nucleic acids encoding these Cas proteins may be introduced into the cell simultaneously or sequentially. For example, the Cas proteins Cas5d, Cas6d and Cas7d synthesized in vitro or in vivo and the gRNA synthesized in vitro or in vivo may be incubated in vitro to form a complex, and the complex may be introduced into the cell.

Upon introduction of the target recognition module and the gRNA, the cell is cultured under suitable conditions for targeting of a target nucleotide sequence. The cell is then cultured under suitable conditions for cell growth and maintenance. The culture conditions may be suitable for the organism species in which the cell into which the target recognition module and the gRNA are introduced is derived from, and can be appropriately determined by a person skilled in the art, for example, based on known cell culture techniques.

According to the method of targeting a target sequence of the present invention, the gRNA binds to Cas7d of the target recognition module to form a complex of the target recognition module and the gRNA. At the same time, the gRNA forms a base pair with the target nucleotide sequence. The target recognition module targets the target nucleotide sequence in a sequence-specific manner by recognizing the PAM sequence present in the vicinity of the target nucleotide sequence. In the target sequence-targeting method of the present invention, Cas10d may be further introduced into the cell.

### (6) The Method of Targeting a Target Sequence of the Present Invention

The target sequence-altering method of the present invention is characterized by introducing the RNA-guided endonuclease and the gRNA into the cell. Specifically, the target sequence-altering method of the present invention is characterized by introducing into the cell (i) Cas3d, Cas5d, Cas6d, Cas7d and Cas10d, or nucleic acids encoding the proteins, and (ii) the gRNA or a DNA encoding the gRNA. The target sequence-altering method of the present invention comprises cleaving a nucleotide sequence targeted by the target sequence-targeting method of the present invention with the polynucleotide cleavage module. The target sequence-altering method of the present invention may be performed in vitro or in vivo. In the present invention, the alteration includes deletion, insertion, and substitution of one or more nucleotides, and a combination thereof.

In the method of altering the target sequence of the present invention, in addition to the RNA-guided endonuclease and the gRNA, a donor polynucleotide may be introduced into the cell. The donor polynucleotide comprises at least one donor sequence that contains alteration desired to be introduced into a target site. The donor polynucleotide may comprise, in addition to the donor sequence, sequences having high homology with the upstream and downstream sequences of the target sequence (preferably, sequences substantially identical to the upstream and downstream sequences of the target sequence) at both ends of the donor sequence. The donor polynucleotide may be a single-stranded or double-stranded DNA. The donor polynucleotide can be appropriately designed by a person skilled in the art based on techniques known in the art.

When the donor polynucleotide is absent in the method of altering a target sequence of the invention, cleavage in the target nucleotide sequence may be repaired by non-homologous end joining (NHEJ). NHEJ is known to be error-prone, and deletion, insertion, or substitution of one or more nucleotides, or a combination thereof may occur during the cleavage repair. Thus, the sequence may be altered at the target sequence site, and thereby frameshift or an immature stop codon is induced to inactivate or knock out the expression of the gene encoded by the target sequence region.

When the donor polynucleotide is present in the method of altering a target sequence of the present invention, the donor sequence of the donor polynucleotide is inserted into the target sequence site or replaces the target sequence site by homologous recombination repair (HDR) of the cleaved target nucleotide sequence. As a result, desired alteration is introduced into the target sequence site.

The RNA-guided endonuclease may be introduced into the cell as an isolated complex comprising Cas5d, Cas6d, Cas7d, Cas3d and Cas10d, or each of Cas5d, Cas6d, Cas7d, Cas3d and Cas10d may be introduced into the cell as an isolated single protein. The RNA-guided endonuclease may be also introduced into the cell as nucleic acids encoding the Cas proteins Cas5d, Cas6d, Cas7d, Cas3d and Cas10d. Examples of the nucleic acid include RNA such as mRNA and DNA.

The DNA encoding the Cas protein may be contained in, for example, a vector, and the DNA sequence is preferably operably linked to a regulatory sequence such as a promoter or a terminator. When the cell into which the RNA-guided endonuclease is introduced is a eukaryotic cell, a nuclear localizing signal sequence is preferably added to the DNA encoding the Cas protein. Two or more or all of the DNAs encoding the Cas proteins Cas3d, Cas5d, Cas6d, Cas7d and Cas10d may be contained in a single vector or may be contained in separate vectors. The number of vectors and the kinds and combinations of Cas proteins encoded by the DNAs to be incorporated into each vector are not limited. When two or mote DNAs encoding the Cas proteins are contained in a single vector, the DNA sequences may be Linked to each other, for example via a sequence encoding a self-cleaving peptide, so as to be polycistronically expressed. The two or more DNAs encoding the Cas proteins may be linked in any order.

The gRNA may be introduced into the cell as an RNA or as a DNA encoding the gRNA. The DNA encoding the gRNA may be contained, for example, in a vector. The DNA sequence is preferably operably linked to a regulatory sequence such as a promoter or a terminator.

The DNAs encoding the Cas proteins and the DNA encoding the gRNA may be contained in the same vector or may be contained in separate vectors. For example, one or more or all of the DNAs encoding Cas3d, Cas5d, Cas6d, Cas7d and Cas10d, and the DNA encoding the gRNA may be contained in a single vector.

The regulatory sequence such as a promoter or a terminator and the nuclear localizing signal sequence are known in the art, and can be appropriately selected depending on the kind of the cell into which the RAN-guided endonuclease and the gRNA are introduced. The vector used for an introduction may be appropriately selected depending on the kind of the cell into which the vector is introduced, and is not particularly limited. Examples of the vector include plasmid vectors, virus vectors, phagemids, cosmids, artificial/mini-chromosomes, and transposons.

The introduction of the RNA-guided endonuclease, the gRNA, and the donor polynucleotide into the cell can be performed by various means known in the art. Examples of such means include transfection, e.g., calcium phosphate-mediated transfection, electroporation, liposome transfection, etc., virus transduction, lipofection, gene gun, microinjection, Agrobacterium method, Agroinfiltration, and PEG-calcium method.

The RNA-guided endonuclease, the gRNA, and the donor polynucleotide may be introduced into the cell simultaneously or sequentially. Cas3d, Cas5d, Cas6d, Cas7d and Cas10d constituting the RNA-guided endonuclease, or nucleic acids encoding these Cas proteins may be introduced into the cell simultaneously or sequentially.

Upon introduction of the RNA-guided endonuclease and the gRNA or the RNA-guided endonuclease, the gRNA and the donor polynucleotide, the cell is cultured under suitable conditions for cleavage at the target sequence site. The cell is then cultured under suitable conditions for cell growth and maintenance. The culture conditions may be suitable for the organism species which the cell into which the RNA-guided endonuclease and the gRNA or the RNA-guided endonuclease, the gRNA and the donor polynucleotide are introduced is derived from, and can be appropriately determined by a person skilled in the art, for example, based on known cell culture techniques.

According to the method of altering a target sequence of the present invention, the gRNA forms a base pair with the target nucleotide sequence, and at the same time, the gRNA interacts with the target recognition module of the RNA-guided endonuclease to guide the RNA-guided endonuclease to the target sequence site. Then, the cleavage module of the RNA-guided endonuclease cleaves the sequence at the target sequence site. When the cleaved sequence is repaired, the target sequence is altered. For example, the method of altering a target sequence of the present invention can be used for an alteration of a target nucleotide sequence on the genome. A double-stranded DNA on the genome is cleaved and then altered at a target site by the method of altering a target sequence of the present invention.

### (7) The Method of Target Gene Suppression of the Present Invention

The target gene expression-suppressing method of the present invention is characterized by introducing the target recognition module (Cas5d, Cas6d and Cas7d) and the gRNA into the cell. Specifically, the method of target gene suppression of the present invention is characterized by introducing into the cell (i) Cas5d, Cas6d and Cas7d, ox nucleic acids encoding the proteins, and (ii) the gRNA or a DNA encoding the gRNA. In the method of target gene suppression of the present invention, at least a part of the target gene sequence is selected as a target nucleotide sequence, and the gRNA containing a sequence complementary to the target sequence is used. The method of target gene suppression of the present invention comprises suppressing the expression of a gene containing the target sequence by binding of a complex of the target recognition module and the gRNA to the target sequence when targeting the target nucleotide sequence by the method of target gene suppression of the present invention. The method of target gene suppression of the present invention may be performed in vitro or in vivo. According to the method of target gene suppression of the present invention, though the target gene sequence is not cleaved, the function of a gene region containing the target sequence or the expression of the gene is inhibited by binding of the complex of the target recognition module and the gRNA to the target nucleotide sequence.

The target recognition module and the gRNA, a method for introducing them into the cell, cell culture at the time of introduction and after introduction, and the like are as described in “(5) The method of targeting a target sequence of the present. invention”. In the method of target gene suppression of the present invention, Cas10d may be further introduced into the cell.

### (8) Complex of the Present Invention

The complex of the present invention comprises the CRISPR type I-D Cas proteins and the gRNA. The present invention particularly provides a complex comprising the target recognition module and the gRNA, and a complex comprising the RNA-guided endonuclease and the gRNA. More specifically, a complex comprising Cased, Cased, Cas7d and the gRNA, and a complex comprising Cas5d, Cas6d, Cas7, Cas3d, Cas10d and the gRNA are provided. In addition, a DNA molecule encoding the complex is provided. The complex of the present. invention can be used in the method of altering a target sequence, the method of target gene suppression and the method of targeting a target sequence of the present invention. A target sequence on the genome of a cell can be altered by introducing a complex comprising the RNA-guided endonuclease (a complex comprising Cas5d, Cas6d, Cas7d, Cas3d and Cas10d) and the gRNA into the cell to allow the complex to function in the cell. In addition, a target sequence in a cell can be targeted and the expression of a gene encoded by a target sequence region can be suppressed by introducing a complex comprising the target recognition module (a complex comprising Cas5d, Cas6d and Cas7d) and the gRNA into the cell to allow the complex to function in the cell. The complex comprising the target recognition module and the gRNA may further contain Cas10d.

The complex of the present invention can be produced in vitro or in vivo by a conventional method. For example, nucleic acids encoding the Cas proteins constituting the RNA-guided endonuclease or the target recognition module, and the gRNA or a DNA encoding the gRNA may be introduced into a cell to allow the complex to form in the cell.

Examples of the complex of the present invention include, but not limited to, a complex comprising Cas5d (SEQ ID NO: 1), Cas6d (SEQ ID NO: 2) and Cas7d (SEQ ID NO: 3) from Microcystis aeruginosa, and a gRNA consisting of a sequence shown by GUUCCAAUUAAUCUUAAGCCCUAUUAGGGAUUGAAACNNNNNNNNNNNNNNNNNNNNNNNNNNNNNNNNN NNGUUCCAAUUAAUCUUAAGCCCUAUUAGGGAUUGAAAC (SEQ ID NO: 6; is any nucleotide constituting a sequence complementary to a target nucleotide sequence), and a complex comprising Cas 5d (SEQ ID NO: 1), Cas6d (SEQ ID NO: 2), Cas7d (SEQ ID NO: 3), Cas3d (SEQ ID NO: 4), and Cas10d (SEQ ID NO: 5) from Microcystis aeruginosa, and a gRNA consisting of a sequence shown by GUUCCAAUUAAUCUUAAGCCCUAUUAGGGAUUGAAACNNNNNNNNNNNNNNNNNNNNNNNNNNNNNNNNN NNGUUCCAAUUAAUCUUAAGCCCUAUUAGGGAUUGAAAC (SEQ ID NO:6; N is any nucleotide constituting a sequence complementary to a target nucleotide sequence). In the gRNA sequence, the number of N may be varied within a range of 10 to 70, preferably 20 to 50, more preferably 25 to 45, still more preferably 30 to 40, and still more preferably 32 to 37.

### (9) Expression Vector of the Present Invention

The present invention further provides an expression vector containing a nucleic acid encoding the RNA-guided endonuclease comprising Cas3d, Cas5d, Cas6d, Cas7d and Cas10d, and a DNA encoding the gRNA comprising a sequence complementary to a target sequence and common repetitive sequences derived from a CRISPR locus, preceding and following the target sequence, and an expression vector containing nucleic acids encoding CRISPR type I-D-associated proteins Cas5d, Cas6d and Cas7d, and a DNA encoding the gRNA comprising a sequence complementary to a target sequence and common repetitive sequences derived from a CRISPR locus, preceding and following the target sequence.

The vector of the present invention is a vector for introducing the Cas proteins and the gRNA into the cell, as described in “(5) Target sequence-targeting method of the present invention”, “(6) Target sequence-altering method of the present invention”, and “(7) Target gene expression-suppressing method of the present invention”. After the introduction of the vector into the cell, the Cas proteins and the gRNA are expressed in the cell. The vector of the present invention may be also a vector in which the target sequence contained in the gRNA is replaced by any sequence containing a restriction site. Such a vector is used after incorporating a desired target nucleotide sequence into the restriction site. Any sequence may be, for example, a spacer sequence present on a CRISPR type I-D locus or a part of the spacer sequence.

### (10) Fusion Protein Comprising the Target Recognition Module of the Present Invention

The present invention further provides a fusion protein comprising the target recognition module and a functional polypeptide. When the fusion protein and the gRNA are introduced into a cell, the fusion protein is guided to a target nucleotide sequence or a target gene in the cell by the action of the target recognition module and the gRNA, and the target nucleotide sequence or the target gene is altered or modified by the action of the functional polypeptide. Thus the present invention further provides a method for altering or modifying a target nucleotide sequence or a target gene, which comprises introducing the fusion protein and the gRNA into a cell. Furthermore, the present invention provides a complex comprising the fusion protein and the gRNA.

The functional polypeptide is a polypeptide that exhibits any function to a target sequence and is a polypeptide other than Cas3d and Cas10d. Examples of the functional polypeptide include, but not limited to, restriction enzymes, transcription factors, DNA methylases, histone acetylases, fluorescent proteins; polynucleotide cleavage modules, for example, nucleotide cleavage modules of restriction enzymes; gene expression regulation modules, for example, transcription activation modules and transcription repression modules of transcription factors; and epigenomic modification modules, for example, methylation modules of DNA methylases, and histone acetylation modules of histone acetylases. An example of the fluorescent protein is GFP. For example, a target sequence can be altered by introducing the fusion protein comprising the target recognition module and the polynucleotide cleavage module together with the gRNA into a cell, in the same manner as the method of altering a target sequence of the present invention. For example, a target sequence can be modified to regulate the expression of a target gene by introducing the fusion protein comprising the target recognition module and the gene expression regulation module or the epigenomic modification module together with the gRNA into a cell. For example, the vicinity of a target sequence can be fluorescently labeled by introducing the fusion protein comprising the target recognition module and the fluorescent protein together with the gRNA into a cell.

Hereinafter, examples of the present invention are shown. However, the present invention is not limited to the examples.

### Examples

As one embodiment, a group of genes (Cas3d, Cas5d, Cas6d, Cas7d, Cas10d) derived from the CRISPR type I-D (hereinafter also referred to as “TiD”) locus derived from Microcystis aeruginosa was cloned and then used. For processing and construction of DNA sequences in Examples, artificial gene chemical synthesis, PCR, restriction enzyme treatment, ligation, or a Gibson Assembly method was used. In addition, the Sanger method or a next generation sequencing method was used to determine nucleotide sequences.

### Example 1. Genome Editing in E. coli

In this Example, it was demonstrated that the technique of the present invention effectively functions in E. coli, which is a typical bacterial model organism.

### (1) Construction of TiD Gene Expression Plasmid

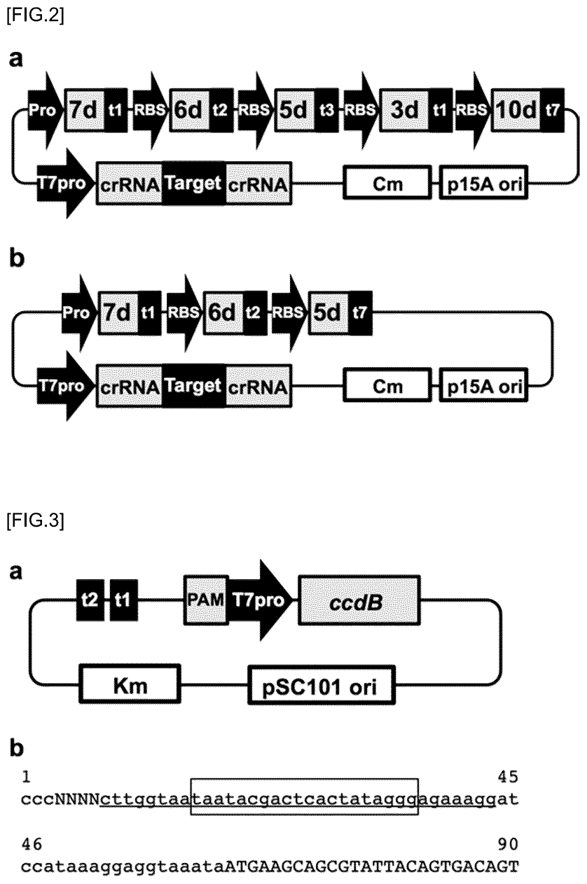

A gene group derived from the CRISPR type I-D locus (hereinafter also referred to as “TiD locus”) of Microcystis aeruginosa (hereinafter also referred to as “M. aeruginosa”) was cloned. E. coli codon-optimized sequences (SEQ ID NOs: 7 to 11) encoding each Cas protein ware artificially chemically synthesized based on the amino acid sequence information of Cas5d, Cas6d, Cas7d, Cas3d and Cas10d derived from the TiD locus from M. aeruginosa. A DNA fragment comprising a J23108 synthetic promoter (SEQ ID NO: 12) or a synthetic ribosome binding sequence (SEQ ID NO: 13) upstream of each Cas protein-encoding gene and a terminator sequence (SEQ ID NO: 14 to 17) downstream of each Cas protein-encoding gene was ligated into plasmid vector pACYC184 (manufactured by Nippon gene) to construct pEcTiD1. In addition, a CRISPR repeat sequence (crRNA, SEQ ID NO: 18) present in the vicinity of the CRISPR type I-D locus from M. aeruginosa was extracted, and a crRNA expression cassette (SEQ ID NO: 20) containing the CRISPR repeat. sequence under the control of a T7 promoter (SEQ ID NO: 19) was synthesized. The crRNA expression cassette contained a promoter region sequence of E. coli code gene which was a target sequence in this Example. The crRNA expression cassette sequence was incorporated into pEcTiD1 to construct pEcTiD2 (FIG. 2a). Moreover, pEcTiD3 containing Cas5d, Cas6d and Cas7d gene expression cassettes was constructed as a TiD expression plasmid vector for genome editing without DNA double-strand break (FIG. 2b). The promoters, terminators, CRISPR repeat sequence, and crRNA expression cassette sequence used in this Example are shown in Table 2.

TABLE 2

| | |
|---|---|
| J23108 | 5′-CTGACAGCTAGCTCAGTCCTAGGTATAATGCTA |
| synthetic | GC-3′ (SEQ ID NO: 12) |
| promoter | |
| ribosome | 5′-AATAATTTTGTTTAACTTTAAGAAGGAGATATAC |
| binding | AT-3′ (SEQ ID NO: 13) |
| sequence | |
| (RBS) | |
| terminator | 5′-AGATCCTGTAAAACGACGGCCAGT-3′ (SEQ ID |
| sequence | NO: 14) |
| STOP767 | |
| terminator | 5′-CGCCAGGGTTTTCCCAGTC-3′ (SEQ ID |
| sequence | NO: 15) |
| STOP768(1) | |
| terminator | 5′-CGCCAGGGTTTTCCCAGTC-3′ (SEQ ID |
| sequence | NO: 16) |
| TOP768(2) | |
| T7 | 5′- |
| terminator | TAGCATAACCCCTTGGGGCCTCTAAACGGGTCTTGAGGGG |
| sequence | TTTTTTG-3′ (SEQ ID NO: 17) |
| CRISPR | 5′- |
| repeat | GTTCCAATTAATCTTAAGCCCTATTAGGGATTGAAAC-3′ |
| sequence | (SEQ ID NO: 18) |
| T7 promoter | 5′-TAATACGACTCACTATAGG-3′ |
| sequence | (SEQ ID NO: 19) |
| crRNA | 5′-GTTCCAATTAATCTTAAGCCCTATTAGGGATTGAAAC |
| expression | ggtaataatacgactcactatagggagaaaggatcGTTCC |
| cassette | AATTAATCTTAAGCCCTATTAGGGATTGAAAC-3′ (SEQ |
| sequence | ID NO: 20) |
| | (Upper case letters indicate TiD CRISPR |
| | repeat sequence crRNA. Lower case |
| | letters indicate a sequence of 35 |
| | nucleotides of the promoter region of |
| | the ccdB gege, which is the target |
| | sequence.) |

### (2) Construction of Protospacer Adjacent Motif (PAM) Library

In this Example, a synthetic ccdB gene cassette (SEQ ID NO: 21) (Table 3) in which the T7 promoter sequence is linked upstream of E. coli code gene as the target DNA was used. The target sequence of TiD was a sequence of 35 nucleotides comprising the T7 promoter region upstream of the ccdB gene. The synthetic ccdB gene cassette was ligated to a multicloning site in plasmid vector pMW219 (manufactured by Nippon gene) to construct pMW_ccdB1 (FIG. 3a).

The CRISPR system recognizes a protospacer adjacent motif (PAM) sequence located in the vicinity of a target sequence and binds to the target sequence via a gRNA. Since the PAM sequence of M. aeruginosa TiD used in this Example was unknown, a PAM sequence library plasmid for determining the PAM sequence of M. aeruginosa TiD was constructed. Random four nucleotide sequences were introduced upstream of the T7 promoter of pMW_ccdB1 by using artificial chemical DNA synthesis and PCR (FIG. 3b). The constructed pMW_ccdB-PAM library plasmids were introduced into a ccdB resistant E. coli cell line retaining the CcdB resistance (manufactured by Thermo Fisher Scientific), and then the plasmids were prepared.

TABLE 3
SEQ ID NO: 21
Synthetic ccdB gene expression cassette (The
underlined sequence indicates the T7 promoter. The
uppercase letters indicate the coding region for
ccdB gene.)
5′-aggctttaatacgactcactatagggagaaaggatccataaaggagg
taaataatgaagcagcgtattacagtgacagttgagacagctatcagttg
ctcaaggcatatatgatgtcaatatctccggtctggtaagcacaaccatg
cagaatgaagcccgtcgtctgccgaacgctggaaagcggaaaatcaggaa
gggatggctgaggtcgcccggtttattgaaatgaacggctcttttgctga
cgaggggactggtgaaATGCAGTTTAAGGTTTACACCTATAAAAGAGAGAG
CCGTTATCGT [symbol] TTTGTGGATGTACAGAGTGATATTATTGACACGCCCGGG
CGACGGATGGTGAT [symbol] CCTGGCCAGTGCACGTCTGCTGTCAGATAAAGTCT
CCCGTGAACTTTACCCGGTGCATATCGGGGATGAAAGCTGGCGCATGATG
ACCACCGATATGGCCAGTGTGGTCTCCGTTATCGGGGAAGAAGTGGCTGA
TCTCAGCCACCGCGAAAATGACA [symbol] AAAACGCCATTAACCTGATGTTCTGG
GGAATATAA-3′ [symbol]
indicates data missing or illegible when filed

### (3) Determination of PAM Sequence in M. aeruginosa TiD System

The PAM sequence for TiD was determined by using pEcTiD3-T7 which has inserted the sequence of 35 nucleotides complementary to the T7 promoter region on the pMW_ccdB-PAM Library plasmid. The pEcTiD3-T7 was introduced into E. coli BL21AI strain (manufactured by Thermo Fisher Scientific) to obtain an E. coli host strain for the ccdB gene genome editing. The BL21AI [pEcTiD3-T7] strain expresses Cas5d, Cas6d and Cas7d proteins necessary for target A sequence recognition. A Cas5d/Cas6d/Cas7d-crRNA complex recognizes a target sequence adjacent to the appropriate PAM sequence and binds to the target sequence to inhibit the function of the T7 promoter, which is the target sequence, though it does not cleave the target sequence.

The ccdB expression by the pMW-ccdB-PAM introduced into the BL21AI strain is induced in an arabinose-supplemented medium, and BL21AI cells not having the CcdB resistance are killed. When the pMW_ccdB-PAM library plasmid is introduced into BL21AI cells into which the TiD expression plasmid has been introduced in advance, the Cas5d/Cas5d/Cas7-crRNA expressed from the pEcTiD3 plasmid binds to the T7 promoter of the pMW_ccdB-PAM library plasmid having an appropriate PAM sequence to be recognized by TiD, thereby production of CcdB protein is inhibited, and thus the E. coli cells can grow. From the grown E. coli colonies, the pMW_ccdB-PAM library plasmid was prepared, and the PAM sequence was analyzed by sequencing to determine the PAM sequence of M. aeruginosa TiD.

The pMW_ccdB-PAM library plasmids, which were prepared in large amounts, were introduced into the BL21AI [pEcTiD3-T7] strain by a chemical competent cell method. The BL21AI cells retaining the pMW_ccdB-PAM library plasmid and pEcTiD3-T7 were selected on an LB agar medium containing 25 mg/L chloramphenicol, 25 mg/L kanamycin and 1% glucose. From E. coli colonies thus obtained, about 1×10⁷ colonies were collected, washed several times with an LB liquid medium not containing antibiotics and glucose, and then suspended in an LB liquid medium containing 1% arabinose at 1×10⁶ cells/mL. The suspension was cultured with shaking at 37° C. for 2 hours to induce the expression of the crRNA and the ccdB under the control of the T7 promoter by arabinose. Then, 200 μl of the suspension was streaked on an LB agar medium containing 25 mg/L chloramphenicol, 25 mg/L kanamycin and 1% arabinose. After culturing overnight at 37° C., bacterial colonies were collected. From about 500 colonies thus collected, their plasmids were prepared, and the vicinity of the PAM sequence was subjected to sequencing analysis. The PAM sequences of the pMW_ccdB-PAM library plasmids rescued in the presence of the TiD expression plasmid contained sequence 5′-NGTH-3′ (N=A, C, G or T; H=A, C or T). The use frequencies of the PAM sequences were 28% for NGTA, 33% for NGTC, and 38% for NGTT. Therefore, it was found that the PAM sequence utilized by TiD was 5′-GTH-3′ (H=A, C or T).

### (4) Genome Editing in E. coli

Plasmids pMW_ccdB-PAMgta, pMW_ccdB-PAMgtc and pMW_ccdB-PAMgtt containing the three types of PAM sequences determined using pEcTiD3-T7 and pMW_ccdB-PAM library plasmids were constructed, and introduced together with pEcTiD2-17 into the BL21AI strain. The BL21AI cells retaining pMW_ccdB-PAMgta/pEcTiC2-T7, pMW_ccdB-PAMgtc/pEcTiD2-T7 and pMW_ccdB-PAMgtt/pEcTiD2-T7 were selected on an LB agar medium containing 25 mg/L chloramphenicol, 25 mg/L kanamycin and 1% glucose, and then found to contain the plasmid introduced into each bacterial cell by sequencing analysis. Subsequently, the BL21AI cells retaining the correct plasmid were streaked on an LB agar medium containing 25 mg/L chloramphenicol, 25 mg/L kanamycin and 1% arabinose and then cultured overnight at 37° C. As a result, all bacterial cells did not grow, which was probably caused by double-strand DNA break on the plasmid DNA in the presence of Cas3d and Cas10d.

### Example 2. Genome Editing in Higher Plants

In this example, as an embodiment of genome editing in higher eukaryotes, it was demonstrated that the technique of the present invention effectively functions in Nicotiana benthamiana and Solanum lycopersicum.

### (1) Construction of Binary Vector for TiD Gene Expression in Higher Plant Cells

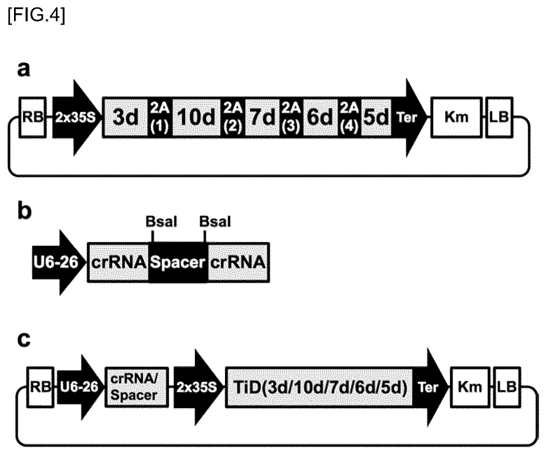

According to frequencies in Arabidopsis and tobacco, dicotyledon codon-optimized sequences encoding each Cas protein were artificially chemically synthesized based on the amino acid sequence information of Cas5d, Cas6d, Cas7d, Cas3d and Cas10d derived from the TiD locus of M. aeruginosa. A DNA fragment comprising a nuclear localizing signal sequence (SEQ ID NO: 22, SEQ ID NO: 23) containing two nuclear localizing signals arranged in tandem 5′-upstream of each of the Cas protein-encoding genes, and a self-cleaving peptide 2A sequence (SEQ ID NOs: 24-28) between the Cas protein-encoding genes was prepared. A promoter sequence (2×35S promoter; SEQ ID NO: 29) comprising tandemly arranged two cauliflower mosaic virus 35S gene promoters and the translation enhancer Ω sequence was linked to the 5′-upstream of the five TiD gene fragments fused to each other via 2A peptide sequences, and the Arabidopsis heat shock protein 18.2 kDa gene terminator sequence (SEQ ID NO: 30) was linked to the 3′-downstream of the five TiD gene fragments fused to each other via the 2A peptide sequences, and thereby a TiD gene expression cassette was prepared. The TiD gene expression cassette was cloned into binary plasmid vector pCAMBIA2300 to construct pEgPTiD1 (FIG. 4a). For a crRNA expression cassette for plants, a DNA in which a spacer sequence containing two restriction enzyme BsaI sites was placed between two crRNA sequences so that any sequence of 35 nucleotides artificially chemically synthesized could be ligated into the BsaI sites (SEQ ID NO: 31). The Arabidopsis U6 snRNA-26 gene promoter sequence (SEQ ID NO: 32) was ligated at 5′-upstream of the crRNA expression cassette sequence, and the poly T sequence was ligated at 3′-downstream of the crRNA expression cassette sequence (FIG. 4b). The crRNA expression cassette for plants was ligated between the RB sequence and the 2×35S promoter. of pEgPTidD1 to construct pEgPTiD2, which was used as a TiD gene expression binary plasmid vector for plant genome editing (FIG. 4c). The dicotyledonous codon-optimized sequences encoding each Cas protein to which a nuclear localizing signal is ligated into pEgPTidD1 and pEgPTidD2 are shown in SEQ ID NOs: 33 to 37. The nuclear localizing signal sequence, self-cleaving peptide 2A sequence, promoter, terminator, and crRNA expression cassette sequence used in this Example are shown in Table 4.

TABLE 4

| | |
|---|---|
| nuclear | DPKKKRKVDPKKKRKVSG (SEQ ID |
| localizing | NO: 22) |
| signal amino | |
| acid sequence | |
| nuclear | 5′- |
| localizing | GACCCAAAGAAGAAGCGGAAGGTAGACC |
| signal sequence | CTAAGAAGAAGCGCAAGGTTTCTGGA-3′ |
| (encoding | (SEQ ID NO: 23) |
| SEQ ID NO: 22) | |
| self-cleaving | GSEGRGSLLTCGDVEENPGP (SEQ ID |
| peptide 2A amino | NO: 24) |
| acid sequence | |
| self-cleaving | 5′- |
| peptide 2A(1) | GGCTCTGAGGGCAGAGGCAGCCTGCTGACCT |
| sequence | GCGGCGACGTGGAGGAAAACCCTGGCCCT- |
| (encoding | 3′ (SEQ ID NO: 25) |
| SEQ ID NO: 24) | |
| self-cleaving | 5′- |
| peptide 2A(2) | GGGTCTGAGGGACGCGGCTCCCTGCTCACCT |
| sequence | GTGGAGATGTGGAAGAGAACCCAGGCCCC- |
| (encoding | 3′ (SEQ ID NO: 26) |
| SEQ ID NO: 24) | |
| self-cleaving | 5′- |
| peptide 2A(3) | GGTTCTGAAGGCAGAGGCTCTCTGCTGACAT |
| sequence | GTGGGGATGTGGAGGAAAATCCTGGCCCT- |
| (encoding | 3′ (SEQ ID NO: 27) |
| SEQ ID NO: 24) | |
| self-cleaving | 5′- |
| peptide 2A(4) | GGATCCGAGGGCAGAGGAAGTCTGCTAACAT |
| sequence | GCGGTGACGTTGAGGAGAATCCCGGGCCA- |
| (encoding | 3′ (SEQ ID NO: 28) |
| SEQ ID NO: 24) | |
| 2 × cauliflower | 5′- |
| mosaic virus 35S | GCCAACATGGTGGAGCACGACACTCTCGTCT |
| gene promoter + | ACTCCAAGAATATCAAAGATACAGTCTCAGA |
| Ω sequence | AGACCAAAGGGCTATTGAGACTTTTCAACAA |
| | AGGGTAATATCGGGAAACCTCCTCGGATTCC |
| | ATTGCCCAGCTATCTGTCACTTCATCAAAAG |
| | GACAGTAGAAAAGGAAGGTGGCACCTACAAA |
| | TGCCATCATTGCGATAAAGGAAAGGCTATCG |
| | TTCAAGATGCCTCTGCCGACAGTGGTCCCAA |
| | AGATGGACCCCCACCCACGAGGAGCATCGTG |
| | GAAAAAGAAGACGTTCCAACCACGTCTTCAA |
| | AGCAAGTGGATTGATGTGAACATGGTGGAGC |
| | ACGACACTCTCGTCTACTCCAAGAATATCAA |
| | AGATACAGTCTCAGAAGACCAAAGGGCTATT |
| | GAGACTTTTCAACAAAGGGTAATATCGGGAA |
| | ACCTCCTCGGATTCCATTGCCCAGCTATCTG |
| | TCACTTCATCAAAAGGACAGTAGAAAAGGAA |
| | GGTGGCACCTACAAATGCCATCATTGCGATA |
| | AAGGAAAGGCTATCGTTCAAGATGCCTCTGC |
| | CGACAGTGGTCCCAAAGATGGACCCCCACCC |
| | ACGAGGAGCATCGTGGAAAAAGAAGACGTTC |
| | CAACCACGTCTTCAAAGCAAGTGGATTGATG |
| | TGATATCTCCACTGACGTAAGGGATGACGCA |
| | CAATCCCACTATCCTTCGCAAGACCCTTCCT |
| | CTATATAAGGAAGTTCATTTCATTTGGAGAG |
| | GCCGGTCTAGAGTATTTTTACAACAATTACC |
| | AACAACAACAAACAACAAACAACATTACAAT |
| | TACTATTTACAATT-3′ (SEQ ID NO: 29) |
| Arabidopsis heat | 5′- |
| shock protein | ATATGAAGATGAAGATGAAATATTTGGTGTG |
| 18.2 kDa gene | TCAAATAAAAAGCTTGTGTGCTTAAGTTTGT |
| terminator | GTTTTTTTCTTGGCTTGTTGTGTTATGAATT |
| | TGTGGCTTTTTCTAATATTAAATGAATGTAA |
| | GATCTCATTATAATGAATAAACAAATGTTTC |
| | TATAATCCATTGTGAATGTTTTGTTGGATCT |
| | CTTCTGCAGCATATAACTACTGTATGTGCTA |
| | TGGTATGGACTATGGAATATGATTAAAGATA |
| | AGATGGGCTCATAGAGTAAAACGAGGCGAGG |
| | GACCTATAAACCTCCCTTCATCATGCTATTT |
| | CATGATCTATTTTATAAAATAAAGATGTAGA |
| | AAAAAGTAAGCGTAATAACCGCAAAACAAAT |
| | GATTTAAAACATGGCACATAATGAGGAGATT |
| | AAGTTCGGTTTACGTTTATTTTAGTACTAAT |
| | TGTAACGTGAGAC-3′ (SEQ ID NO: 30) |
| crRNA expression | 5′- |
| cassette for | GTTCCAATTAATCTTAAGCCCTATTAGGGAT |
| plants | TGAACggagaccctcaattgtcggtctcGTT |
| | CCAATTAATCTTAAGCCCTATTAGGGATTGA |
| | AAC-3′ (SEQ ID NO: 31) |
| Arabidopsis | 5′- |
| thaliana U6 | AAGCTTCGTTGAACAACGGAAACTCGACTTG |
| snRNA-26 gene | CCTTCCGCACAATACATCATTTCTTCTTAGC |
| promoter sequence | TTTTTTTCTTCTTCTTCGTTCATACAGTTTT |
| | TTTTTGTTTATCAGCTTACATTTTCTTGAAC |
| | CGTAGCTTTCGTTTTCTTCTTTTTAACTTTC |
| | CATTCGGAGTTTTTGTATCTTGTTTCATAGT |
| | TTGTCCCAGGATTAGAATGATTAGGCATCGA |
| | ACCTTCAAGAATTTGATTGAATAAAACATCT |
| | TCATTCTTAAGATATGAAGATAATCTTCAAA |
| | AGGCCCCTGGGAATCTGAAAGAAGAGAAGCA |
| | GGCCCATTTATATGGGAAAGAACAATAGTAT |
| | TTCTTATATAGGCCCATTTAAGTTGAAAACA |
| | ATCTTCAAAAGTCCCACATCGCTTAGATAAG |
| | AAAACGAAGCTGAGTTTATATACAGCTAGAG |
| | TCGAAGTAGTGATT-3′ (SEQ ID NO: 32) |

### (2) Genome Editing in Nicotiana benthamiana

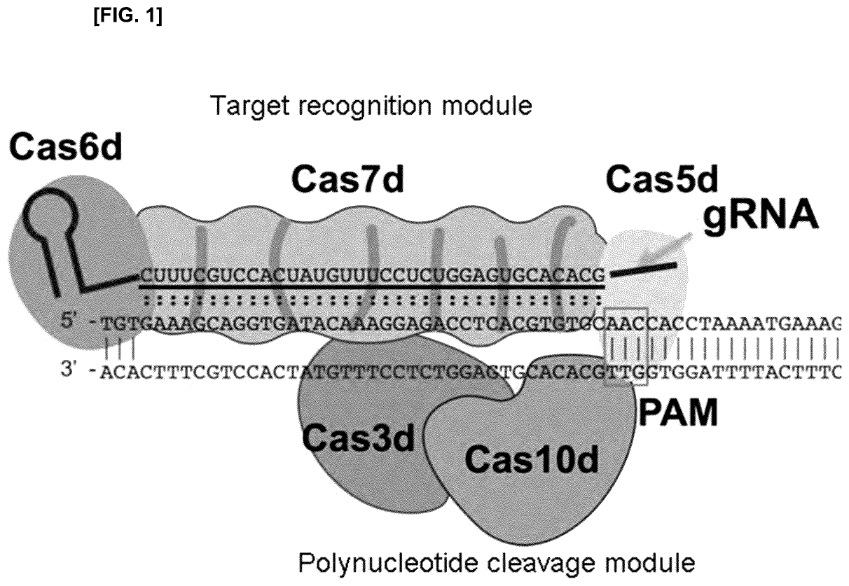

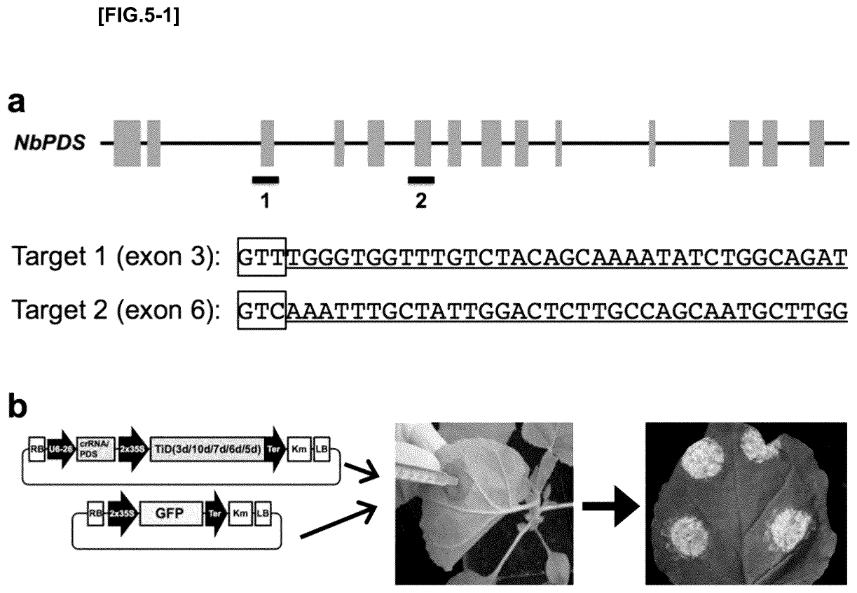

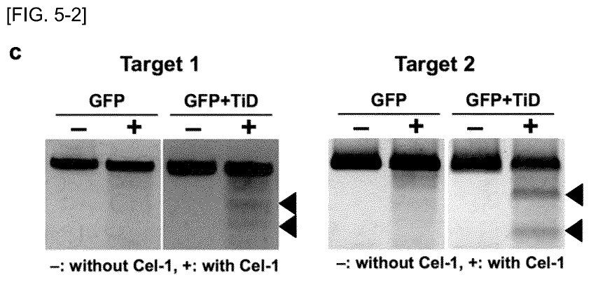

In Example of tobacco, the phytoene desaturase (PDS) gene was selected as a target sequence for introduction of mutations (FIG. 5-1 a). Target sequence 1 (Target 1, SEQ ID NO: 38) was selected from the third exon in the tobacco PDS gene, and the artificial chemical synthesized DNA of target 1 was ligated into the crRNA expression cassette for plants to construct pEgPTiD2-pds (1). Similarly, target sequence 2 (Target 2, SEQ ID NO: 39) was selected from the sixth exon, and the artificial chemical synthesized DNA of target 2 was ligated into the crRNA expression cassette for plants to construct pEgPTiD2-pds(2). The binary vectors thus constructed were introduced into Agrobacterium tumefaciens strain GV2260. Introduction of the TiD expression vector targeting tobacco PDS into tobacco cells was performed by agroinfiltration. Agrobacterium cells retaining pEgPTiD2-pds(1) or pEgPTidD2-pds(2) and Agrobacterium cells retaining a GFP expression binary vector were separately cultured, and then co-infected to the true leaf of Nicotiana benthamiana (FIG. 5-1 b). Three days after the co-infection, a genomic DNA was prepared from a region emitting GFP fluorescence in a leaf disc, and then used as a template to PCR-amplify a 300-500 bp PDS gene fragment containing the target sequence. The PCR amplified fragment was used for the Cel-1 assay to analyze whether a mutation was introduced into the PDS gene. As a control, a tobacco leaf disc into which only the GFP expression binary vector was introduced was used. When only the GFP expression vector was introduced, no mutation was observed on the PDS gene. In contrast, when pEgPTiD2-pds and the GFP expression vector were simultaneously introduced, the introduction of a mutation(s) was observed on each target sequence of the PDS gene (FIG. 5-2 c). Target sequences 1 and 2 are shown in Table 5.

TABLE 5

| | |
|---|---|
| Target sequence 1 on | 5′- |
| tobacco PDS gene | TGGGTGGTTTGTCTACAGCAAAATATC |
| (Target 1) | TGGCAGAT-3′ (SEQ ID NO: 38) |
| Target sequence 2 on | 5′- |
| tobacco PDS gene | AAATTTGCTATTGGACTCTTGCCAGCA |
| (Target 2) | ATGCTTGG-3′ (SEQ ID NO: 39) |

### (3) Genome Editing in Solanum lycopersicum

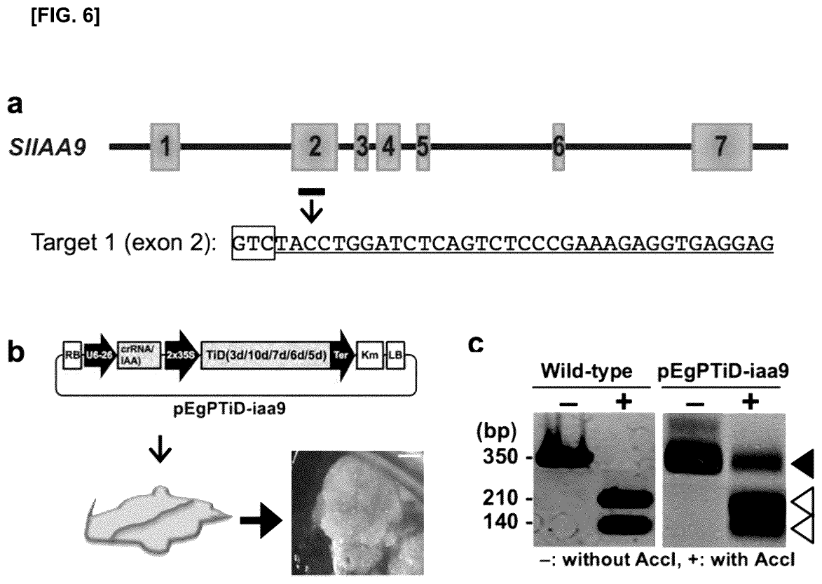

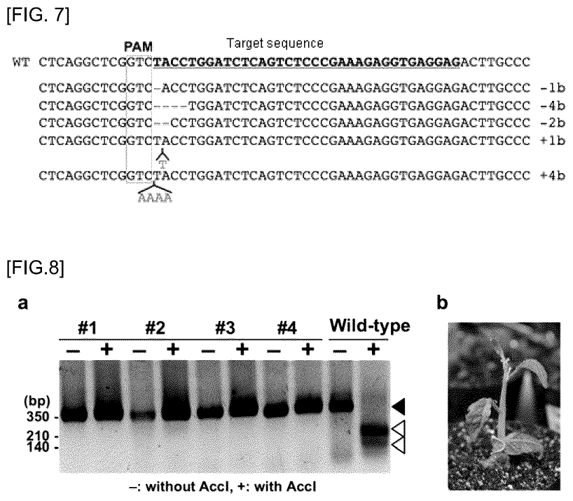

In Example of tomato, an Aux/IAA transcription factor IAA9 gene was selected as a target sequence for introduction of mutations (FIG. 6a). Target sequence 1 (SEQ ID NO: 40) (Table 6) was selected from the second exon of the tomato IAA9 gene, and the artificial chemical synthesized UNA of target 1 was ligated into the crRNA expression cassette for plants to construct pEgPTiD2-iaa9. The constructed binary vector was introduced into Agrobacterium tumefaciens strain GV2260. Introduction of the TiD expression vector targeting the tomato IAA9 gene into tomato cells was performed by an Agrobacterium method using a leaf disc derived from tomato cotyledons. Leaf discs co-inoculated with Agrobacterium was cultured on an MS solidified medium containing 100 mg/L kanamycin and 1.5 mg/L t-zeatin to obtain calli in which gene introduction of a T-DNA region on pEgPTiD2-iaa9 occurred (FIG. 6b). The recognition sequence for the restriction enzyme AccI exists in the target sequence of IAA9. When a mutation is introduced as a result of genome editing by TiD, the AccI recognition site is disappeared. Thus PCR-restriction enzyme length polymorphism (RFLP) analysis using AccI was performed to analyze mutations that occurred on the target sequence of IAA9. A genomic DNA was prepared from the obtained transformed calli and used as a template to PCR-amplify a region of approximately 300 bases containing the target sequence of IAA9. The PCR fragment was digested with AccI. It was found that the PCR fragment from the callus cultures into which pEgPTiD2-iaa9 was introduced contained a sequence that was not digested by AccI as a result of mutation introduction in the IAA9target sequence (FIG. 6c). The nucleotide sequence of the PCR fragment from the callus into which pEgPTiD2-iaa9 was introduced was determined. As a result, it was found that nucleotide deletion of 1 to 4 nucleotides was introduced immediately following the PAM sequence on the target sequence of IAA9. (FIG. 7).

Calli into which pEgPTiD2-iaa9 was introduced was further cultured on an MS solidified medium containing 100 mg/L kanamycin and 1.0 mg/L t-zeatin to obtain transformed and regenerated shoots. A genomic DNA was prepared from the regenerated shoots, and used as a template to perform PCR-RFLP analysis with AccI. As shown in FIG. 8a, PCR fragments that were not cleaved with AccI were observed. In other words, the transformed and regenerated shoots into which the IAA9 target sequence was almost 100% mutated were obtained. Of 14 transformed and regenerated shoots, the 13 shoots showed the same results as shown in FIG. 8a. In these regenerated plants, the true leaves were single leaf-shaped, which is one of phenotypes caused by the deficiency of IAA9. Thus, it was shown that a mutation(s) can be introduced with high efficiency by genome editing using TiD.

TABLE 6

| | |
|---|---|
| Target sequence on | 5′- |
| tomato IAA9 gene | TACCTGGATCTCAGTCTCCCGAAAGAGG |
| | TGAGGAG-3′ (SEQ ID NO: 40) |

### Example 3. Genome Editing in Higher Animals

In this Example, as an embodiment of genome editing in higher animals, it was demonstrated that the technique of the present invention effectively functions in human embryonic kidney cell-derived cell line HEK293.

### (1) Construction of Vector for TiD Gene Expression in Higher Animal Cells

Gene sequences encoding each Cas protein were artificially chemically synthesized based on the amino acid sequence information of Cas5d, Cas6d, Cas7d, Cas3d and Cas10d derived from the TiD locus from M. aeruginosa. A DNA fragment comprising a nuclear localizing signal sequence (SEQ ID NO: 22, SEQ ID NO: 23) containing two nuclear localizing signals arranged in tandem 5′-upstream of each of the Cas protein-encoding genes, and a the self-cleaving peptide 2A sequence (SEQ ID NOS: 24-28) between the Cas protein-encoding genes was prepared. A cytomegalovirus enhancer+chicken β-actin gene promoter hybrid sequence (CBh promoter; SEQ ID NO: 41) was ligated at 5′-upstream of the five TiD gene fragments fused to each other via the 2A peptide sequences, and a bovine growth hormone gene terminator sequence (bGH terminator; SEQ ID NO: 42) was ligated at 3′-downstream of the five TiD gene fragments fused to each other via the 2A peptide sequences, and thereby a TiD gene expression cassette was prepared. The TiD gene expression cassette was ligated into a pCR8TOPO vector (manufactured by Thermo Fisher Scientific) to construct pCR_hTiD. For a crRNA expression cassette, a DNA in which a spacer sequence containing two restriction enzyme BsaI sites was placed between two crRNA sequences so that any sequence of 35 nucleotides could be ligated was artificially chemically synthesized (SEQ ID NO: 31). The human U6 snRNA gene promoter sequence (SEQ ID NO: 43) as an expression control sequence was ligated at 5′-upstream of the crRNA expression cassette, and the poly T sequence was ligated at 3′-downstream of the crRNA expression cassette. The crRNA expression cassette with the human U6 snRNA gene promoter and the poly T sequence was Ligated into a pCR8TOPO vector (manufactured by Thermo Fisher Scientific) to construct pCR_crRNA. The sequences encoding each Cas protein with the nuclear localizing signals in pCR_hTiD are shown as SEQ ID NOs: 33-37. The CBh promoter, bGH terminator, and human U6 snRNA gene promoter sequences are shown in Table 7.

TABLE 7

| | |
|---|---|
| cytomegalo- | 5′- |
| virus | CGTTACATAACTTACGGTAAATGGCCCGCCTGGCTGAC |
| enhancer + | CGCCCAACGACCCCCGCCCATTGACGTCAATAGTAACG |
| universal | CCAATAGGGACTTTCCATTGACGTCAATGGGTGGAGTA |
| chicken β- | TTTACGGTAAACTGCCCACTTGGCAGTACATCAAGTGT |
| actin gene | ATCATATGCCAAGTACGCCCCCTATTGACGTCAATGAC |
| hybrid | GGTAAATGGCCCGCCTGGCATTGTGCCCAGTACATGAC |
| promoter | CTTATGGGACTTTCCTACTTGGCAGTACATCTACGTAT |
| | TAGTCATCGCTATTACCATGGTCGAGGTGAGCCCCACG |
| | TTCTGCTTCACTCTCCCCATCTCCCCCCCCTCCCCACC |
| | CCCAATTTTGTATTTATTTATTTTTTAATTATTTTGTG |
| | CAGCGATGGGGGCGGGGGGGGGGGGGGGGCGCGCGCCA |
| | GGCGGGGCGGGGCGGGGCGAGGGGCGGGGCGGGGCGAG |
| | GCGGAGAGGTGCGGCGGCAGCCAATCAGAGCGGCGCGC |
| | TCCGAAAGTTTCCTTTTATGGCGAGGCGGCGGCGGCGG |
| | CGGCCCTATAAAAAGCGAAGCGCGCGGCGGGCGGGAGT |
| | CGCTGCGACGCTGCCTTCGCCCCGTGCCCCGCTCCGCC |
| | GCCGCCTCGCGCCGCCCGCCCCGGCTCTGACTGACCGC |
| | GTTACTCCCACAGGTGAGCGGGCGGGACGGCCCTTCTC |
| | CTCCGGGCTGTAATTAGCTGAGCAAGAGGTAAGGGTTT |
| | AAGGGATGGTTGGTTGGTGGGGTATTAATGTTTAATTA |
| | CCTGGAGCACCTGCCTGAAATCACTTTTTTTCAGGTTG |
| | GACCGGTGCCACC-3′ (SEQ ID NO: 41) |
| bovine | 5′ |
| growth | GCTGATCAGCCTCGACTGTGCCTTCTAGTTGCCAGCCA |
| hormone | TCTGTTGTTTGCCCCTCCCCCGTGCCTTCCTTGACCCT |
| gene | GGAAGGTGCCACTCCCACTGTCCTTTCCTAATAAAATG |
| terminator | AGGAAATTGCATCGCATTGTCTGAGTAGGTGTCATTCT |
| sequence | ATTCTGGGGGGTGGGGTGGGGCAGGACAGCAAGGGGGA |
| | GGATTGGGAAGAGAATAGCAGGCATGCTGGGGA-3′ |
| | (SEQ ID NO: 42) |
| human U6 | 5′- |
| snRNA gene | GAGGGCCTATTTCCCATGATTCCTTCATATTTGCATAT |
| promoter | ACGATACAAGGCTGTTAGAGAGATAATTGGAATTAATT |
| | TGACTGTAAACACAAAGATATTAGTACAAAATACGTGA |
| | CGTAGAAAGTAATAATTTCTTGGGTAGTTTGCAGTTTT |
| | AAAATTATGTTTTAAAATGGACTATCATATGCTTACCG |
| | TAACTTGAAAGTATTTCGATTTCTIGGCTTTATATATC |
| | TTGTGGAAAGGACGAAACACC-3′ (SEQ ID NO: 43) |

### (2) Genome Editing in Cultured Animal Cells

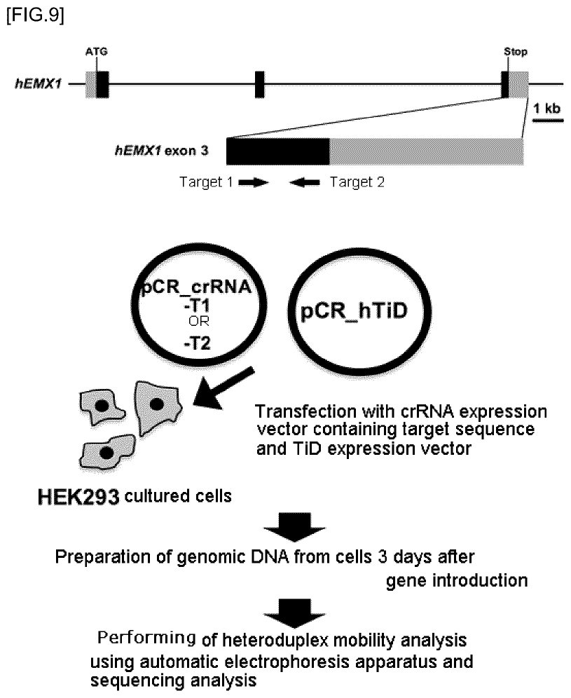

In Example of cultured animal cells, a human embryonic kidney cell-derived cell line (HEK293 cell line) was used, and the EMX1 gene was selected as a target sequence for introduction of mutations. Target 1 (SEQ ID NO: 44) and target 2 (SEQ ID NO: 45) ware selected as target sequences in the EMX1 gene, and the artificial chemical synthesized DNA of target 1 and 2 were ligated into the crRNA expression cassette for cultured human cells prepared in above (1) to construct pUC_crRNA-T1 containing target 1 and pUC_crRNA-12 containing target 2. Constructed plasmids were amplified in E. coli strain HST08 (manufactured by Takara Bio Inc.) and then purified using PureYield (registered trademark) Plasmid Miniprep System (manufactured by Promega Corp.). Among the purified plasmids, a mixture of pCR_hTiD and pUC_crRNA-T1 or a mixture of pCR_NTiD and pUC_crRNA-T2 was introduced into HEK293 cells by transfection. Three days after the introduction of the plasmid vectors, the cells were collected, and a genomic DNA was prepared from them using Blood & Cell Culture DNA Mini Kit (manufactured by Qiagen). Using the genomic DNA thus prepared as a template, the genomic sequence region Comprising target 1 or target 2 was amplified by PCR, and mutation analysis was performed by the heteroduplex mobility analysis using an automatic electrophoresis apparatus MultiNA (manufactured by Shimadzu Corporation). Further, the amplified PCR fragment was cloned into pNEB193 vector (manufactured by New England Biolab), and a mutated sequence was identified by sequencing analysis. Somatic mutation efficiency was calculated based on “the number of clones in which a mutated sequence was observed/the total number of clones analyzed”. The cell line into which the plasmid had not been introduced, or pCR_hTiD, pUC_crRNA-T1 or pUC_crRNA-T2 had been introduced alone was used as a control to perform mutation analysis in the same manner. An experimental scheme for genome editing using the HEK293 cell Line is shown in FIG. 9.

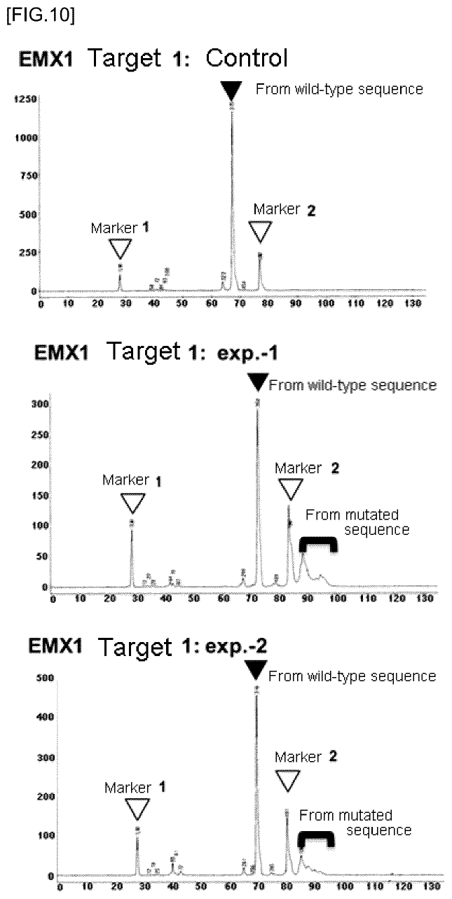

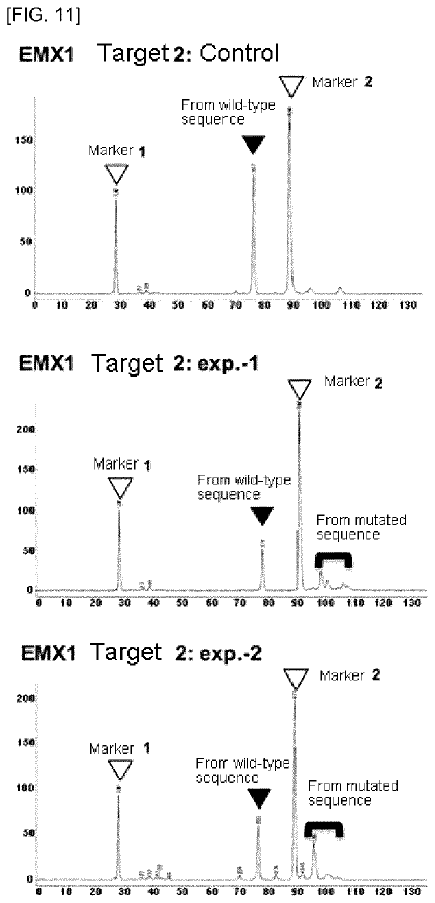

FIG. 10 and FIG. 11 show experimental results obtained when the HEK293 cell line was transfected with the mixture of pCR_hTiD and pUC_crRNA-T1 or the mixture of pCR_hTiD and pUC_crRNA-T2 or when the HEK293 cell line was not transfected with the plasmid (control). As shown in FIG. 10 and FIG. 11, peaks indicating mutations introduced on the target sequence were detected in the HEK293 cell line transfected with the mixture of pCR_hTiD and pUC_crRNA-T1 or the mixture of pCR_hTiD and pUC_crRNA-T2. On the other hand, no peak indicating mutation introduction was detected in the cell line into which the plasmid had not been introduced as a control. Similarly to the cell line into which the plasmid had not been introduced, no peak indicating mutation introduction was detected in the cell line into which pCR_hTiD, pUC_crRNA-T1 or pUC_crRNA-T2 had been introduced alone.

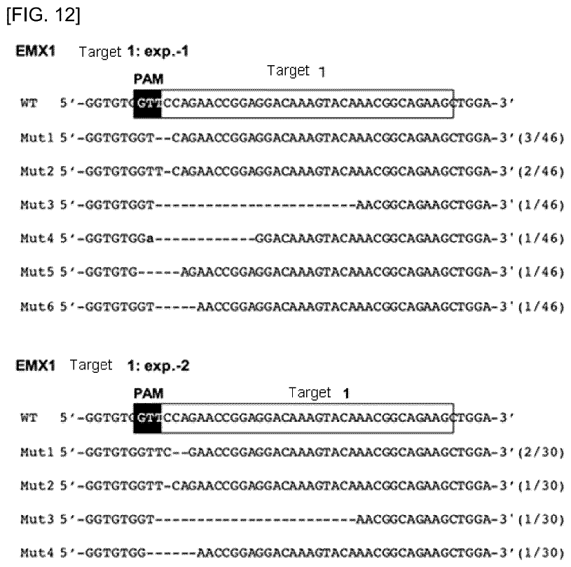

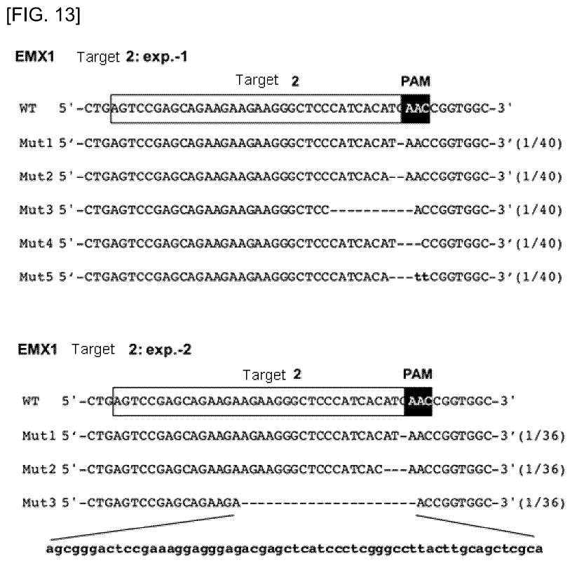

Then, sequence samples in which a peak indicating mutation introduction was detected by heteroduplex mobility analysis were cloned into a plasmid vector and analyzed by sequencing. As a result, as shown in FIG. 12 and FIG. 13, it was found that deletion and/or insertion mutations were introduced on target1 and target 2.

TABLE 8
Target sequences on human EMX 1 gene

| | |
|---|---|
| Target 1 | 5′-CCAGAACCGGAGGACAAAGTACAAACGGCAGAAGC-3′ |
| | (SEQ ID NO: 44) |
| Target 2 | 5′-GATGTGATGGGAGCCCTTCTTCTTCTGCTCGGACT-3′ |
| | (SEQ ID NO: 45) |

### Industrial Applicability

According to the present invention, it is possible to target a gene sequence that cannot be targeted by the conventional genome editing technique using CRISPR type II or type V-derived RNA-guided endonuclease. Specifically, according to the present invention, it is possible to generate mutant alleles, control gene expression by transcriptional activation and inactivation, and realize epigenomic alteration by targeting of a DNA-modifying/histone-modifying protein domain, on gene regions that cannot be targeted by the conventional techniques.

### Sequence Listing Free Text

SEQ ID NO: 1; Microcystis aeruginosa Cas5d amino acid sequence
SEQ ID NO: 2; Microcystis aeruginosa Cas6d amino acid sequence
SEQ ID NO: 3; Microcystis aeruginosa Cas7d amino acid sequence
SEQ ID NO: 4; Microcystis aeruginosa Cas3d amino acid sequence
SEQ ID NO: 5; Microcystis aeruginosa Cas10d amino acid sequence
SEQ ID NO: 6; TiDcrRNA containing direct repeat (37b) and spacer (35b of N). N is any nucleotides constituting a complementary sequence to a target nucleotide sequence.
SEQ ID NO: 7; Cas5d nucleotide sequence for expression in Escherichia coli
SEQ ID NO: 8; Cas6d nucleotide sequence for expression in Escherichia coli
SEQ ID NO: 9; Cas7d nucleotide sequence for expression in Escherichia coli
SEQ ID NO: 10; Cas3d nucleotide sequence for expression in Escherichia coli
SEQ ID NO: 11; Cas10d nucleotide sequence for expression in Escherichia coli
SEQ ID NO: 12; J23108 synthesis promoter
SEQ ID NO: 13; Ribosomal binding sequence
SEQ ID NO: 14; Terminator sequence STOP767
SEQ ID NO: 15; Terminator sequence STOP768 (1)
SEQ ID NO: 16; Terminator sequence TOF768 (2)
SEQ ID NO: 17; T7 terminator sequence
SEQ ID NO: 18; CRISPR repeat sequence
SEQ ID NO: 19; T7 promoter sequence
SEQ ID NO: 20; crRNA expression cassette
SEQ ID NO: 21; Synthesis cccdB gene expression cassette
SEQ ID NO: 22; Nuclear localizing signal (NLS) amino acid sequence
SEQ ID NO: 23; NLS nucleotide sequence
SEQ ID NO: 24; Self-cleaving peptide 2A amino acid sequence
SEQ ID NO: 25; Self-cleaving peptide 2A(1) coding sequence
SEQ ID NO: 26; Self-cleaving peptide 2A(2) coding sequence
SEQ ID NO: 27; Self-cleaving peptide 2A(3) coding sequence
SEQ ID NO: 28; Self-cleaving peptide 2A(4) coding sequence
SEQ ID NO: 29; 2×cauliflower mosaic virus 35S gene promoter+omega sequence
SEQ ID NO: 30; Arabidopsis shock protein 18.2 kDa gene terminator
SEQ ID NO: 31; crRNA expression cassette
SEQ ID NO: 32; Arabidopsis U6 snRNA-26 gene promoter sequence

### SEQ ID NO: 33; 2% NLS+Cas5d

### SEQ ID NO: 34; 2×NLS+Cas6d

### SEQ ID NO: 35; 2×NLS+Cas7d

### SEQ ID NO: 36; 2×NLS+Cas3d

### SEQ ID NO: 37; 2×NLS+Cas10d

SEQ ID NO: 38; Target sequence 1 on tobacco PDS gene
SEQ ID NO: 39; Target sequence 2 on tobacco PDS gene
SEQ ID NO: 40; Target sequence on tomato IAA9 gene
SEQ ID NO: 41; Cytomegalovirus enhancer+universal chicken beta-actin gene hybrid promoter
SEQ ID NO: 42; Bovine-derived growth hormone gene terminator sequence
SEQ ID NO: 43; Human U6 snRNA gene promoter
SEQ ID NO: 44; Target 1 sequence on human EMX1 gene
SEQ ID NO: 45; Target 2 sequence on human EMX1 gene

1. A method for producing a cell comprising an altered target nucleotide sequence, the method comprising:
altering the target nucleotide sequence by the method comprising introducing into a cell:
(i) CRISPR type I-D associated proteins Cas3d, Cas5d, Cas6d, Cas7d and Cas10d, or nucleic acids encoding the proteins, and
(ii) a guide RNA comprising a sequence complementary to the target nucleotide sequence, and common repetitive sequences derived from a CRISPR locus, preceding and following the complementary sequence, or a DNA encoding the guide RNA
wherein the target nucleotide sequence is a double-stranded DNA.

2. The method according to claim 1, wherein the cell is a plant cell.

3. The method according to claim 1, wherein the cell is an animal cell.

4. A method for producing a plant comprising an alteration of a target nucleotide sequence, the method comprising producing a plant cell comprising an altered target nucleotide sequence by the method according to claim 2.

5. A method for producing a non-human animal comprising an alteration of a target nucleotide sequence, the method comprising producing a non-human animal cell comprising an altered target nucleotide sequence by the method according to claim 3.
